(19) United States
(12) Patent Application Publication
(10) Pub. No.: US 2024/0174991 A1
(43) Pub. Date: May 30, 2024

(54) FUSION RT VARIANTS FOR IMPROVED PERFORMANCE

(71) Applicant: 10x Genomics, Inc., Pleasanton, CA (US)

(72) Inventors: Shankar SHASTRY, San Francisco, CA (US); Derek Hunter VALLEJO, San Francisco, CA (US)

(73) Assignee: 10x Genomics, Inc., Pleasanton, CA (US)

(21) Appl. No.: 18/384,537

(22) Filed: Oct. 27, 2023

Related U.S. Application Data
(63) Continuation of application No. PCT/US22/27024, filed on Apr. 29, 2022.
(60) Provisional application No. 63/182,225, filed on Apr. 30, 2021.

(51) Int. Cl. C12N 9/12; C12N 15/10

(52) U.S. Cl. CPC C12N 9/1276; C12N 15/1096; C07K 2319/80

(57) ABSTRACT

The application provides compositions including engineered fusion reverse transcriptases with at least one altered reverse-transcriptase related activity. The engineered fusion reverse transcriptases or reverse transcription enzymes unexpectedly exhibit one or more altered reverse transcriptase related activities such as but not limited to altered template switching efficiency, altered transcription efficiency or both.

### CROSS REFERENCE

This application is a continuation of International Patent Application No. PCT/US2022/027024, filed Apr. 29, 2022, which claims priority to, and benefit of U.S. Provisional Patent Application No. 63/182,225 titled “Fusion RT Variants for Improved Performance” filed on Apr. 30, 2021, the entire disclosures of which are hereby incorporated by reference for all purposes.

### SEQUENCE LISTING

The instant application contains a Sequence Listing which has been submitted electronically in XML format and is hereby incorporated by reference in its entirety. Said XML copy, created on Feb. 6, 2024, is named 131488_0196_Sequence_Listing.xml and is 28 bytes in size.

### FIELD OF INVENTION

The present invention relates to the field of protein engineering, particularly development of reverse transcriptase variants. The reverse transcriptase variants exhibit one or more improved properties of interest.

### BACKGROUND

One of the major challenges in cDNA synthesis reactions is interference in cDNA synthesis from RNA secondary structures. While a higher reaction temperature can remove secondary structure from the template RNA, elevated temperatures typically lead to lower reverse-transcriptase (RT) enzyme activity without the use of an efficient, thermostable RT enzyme. Wild-type (WT) Moloney Murine Leukemia Virus (MMLV) reverse-transcriptase is an RT enzyme that is typically inactivated at higher temperatures. RT enzyme activity can also be reduced by inhibitors, such as inhibitors that might be present in cell lysates, associated reagents and fixation reagents. Low volume reactions can also negatively impact wild-type (WT) MMLV reverse-transcriptase activity.

Specific residues of MMLV have been linked to thermostability. M39V, M66L, E69K, E302R, T306K, W313F, L/K435G, and N454K sites have been shown to improve thermostability, see Arezi et al (2009) Nucleic Acids Res. 37(2):473-481, U.S. Pat. No. 7,078,208, and Baranauskas et al 2012 Prot Engineering 25(10): 657-668, which are hereby incorporated by reference in their entireties.

A wide variety of different applications used in cell processing and analysis methods and systems are known in the art, including but not limited to, analysis of specific individual cells, analysis of different cell types within populations of differing cell types, spatial transcriptomics tissue analysis, analysis and characterization of large populations of cells for environmental, human health, epidemiological and forensic applications. Many of these methods involve the use of a template switching oligonucleotide and require template switching activity.

### SUMMARY

Engineered fusion reverse transcriptases with altered reverse transcriptase-related activities are provided. The engineered fusion reverse transcriptases of the current application exhibit altered reverse transcriptase related activity as compared to a reverse transcriptase having the amino acid sequence set forth in SEQ ID NO:1.

Embodiments of the application provide an engineered fusion reverse transcriptase comprising at least one DNA binding domain (DBD) selected from the group of DNA binding domains comprising archaeal DNA binding domains and single-stranded DNA binding domains and an engineered reverse transcriptase having an amino acid sequence that is at least 90% identical to SEQ ID NO:1 wherein the engineered reverse transcriptase comprises an M39 mutation, a K47 mutation, an L435 mutation, a D449 mutation, a D524 mutation, an E607 mutation, an D653 mutation, and an L671 mutation as indexed to SEQ ID NO:7. In one embodiment, the engineered fusion reverse transcriptase exhibits an altered reverse transcriptase related activity as compared to a reverse transcriptase having the amino acid sequence set forth in SEQ ID NO:1. In various aspects, the at least one DNA binding domain is located at the C-terminus or N-terminus of the engineered fusion reverse transcriptase amino acid sequence.

In certain aspects, the amino acid sequence of the DNA binding domain (DBD) comprises a DNA binding domain comprising SEQ ID NO:2. In various aspects, the DBD is an archaeal DNA binding domain selected from the group comprising Sto7d, Sso7d, Sis7b, Sis7a, Ssh7b, Sto7, Aho7C, Aho7B, Aho7A, Mcu7, Mse7, Sac7e, and Sac7d. In some aspects, the DNA binding domain is a single-stranded DNA binding domain.

In some aspects, the DNA binding domain exhibits reduced RNAase activity. In various aspects, the amino acid sequence of the DNA binding domain has been altered to reduce RNAase activity. The alteration to the amino acid sequence of the DNA binding domain may be selected from the group of alterations comprising a K13 mutation, a K13L mutation, a D36 mutation, and a D36L mutation.

In some aspects, the amino acid sequence of the engineered fusion reverse transcriptase comprises a Sto7 DNA binding domain at the C-terminus of the engineered fusion reverse transcriptase. In one aspect, the amino acid sequence of the engineered reverse transcriptase comprises an amino acid sequence selected from the group of amino acid sequences set forth in SEQ ID NO:3, SEQ ID NO:5, SEQ ID NO:6, and SEQ ID NO:8.

In various aspects of the engineered fusion reverse transcriptase, the amino acid sequence of the engineered reverse transcriptase may further comprise one or both of M39V mutation and an M66L mutation, wherein the mutation is indexed to the amino acid sequence of a wild-type MMLV is set forth in SEQ ID NO:7.

In various aspects of the engineered fusion reverse transcriptases provided herein, the altered reverse transcriptase related activity is selected from the group of reverse transcriptase activities comprising processivity, template switching efficiency and chemical tolerance. In an aspect, the altered reverse transcriptase related activity is an altered template switching (TS) efficiency as compared to the template switching efficiency of a reverse transcriptase having the amino acid sequence set forth in SEQ ID NO:1. In various aspects, the altered template switching efficiency is at least 0.5× greater than the template switching efficiency exhibited by an engineered reverse transcriptase having the amino acid sequence set forth in SEQ ID NO:1.

In various aspects, the engineered fusion reverse transcriptase comprises at least two fusion domains. In certain aspects, at least one fusion domain is located at the N-terminus of the amino acid sequence and at least one fusion domain is located at the C-terminus of the amino acid sequence. In some aspects, at least two fusion domains are located at the same terminus of the amino acid sequence. In some aspects, the fusion domain located at the N-terminus of the amino acid sequence is the same fusion domain located at the C-terminus of the amino acid sequence. In an aspect, the fusion domain located at the N-terminus of the amino acid sequence is Sso7d and the fusion domain located at the C-terminus of the amino acid sequence is Sso7d. In an aspect, the fusion domain located at the N terminus is Sso7d while the fusion domain at the C-terminus is Sto7. In an aspect the fusion domain located at the N-terminus of the amino acid sequence is Sto7 and the fusion domain located at the C-terminus of the amino acid sequence is Sto7. In an aspect the fusion domain located at the N-terminus is Sto7 while the fusion domain at the C-terminus is Sso7d.

The engineered fusion reverse transcriptases provided herein exhibit an altered reverse transcriptase related activity. In various aspects, the altered reverse transcriptase related activity is an increased transcription efficiency as compared to the transcription efficiency of a reverse transcriptase having the amino acid sequence set forth in SEQ ID NO:1. In various aspects the altered reverse transcriptase related activity is an increased transcription efficiency and an increased template switching efficiency as compared to a reverse transcriptase having the amino acid sequence set forth in SEQ ID NO:1. In some aspects, the altered reverse transcriptase related activity is an altered processivity as compared to the processivity of a reverse transcriptase having the amino acid sequence set forth in SEQ ID NO:1. In certain aspects the altered reverse transcriptase related activity is an increase in mitochondrial UMI counts as compared to the mitochondrial UMI counts of a reverse transcriptase having the amino acid sequence set forth in SEQ ID NO:1. In various aspects, the altered reverse transcriptase related activity is an increase in ribosomal UMI counts as compared to the ribosomal UMI counts of a reverse transcriptase having the amino acid sequence set forth in SEQ ID NO:1. In aspects the altered reverse transcriptase related activity is an increased ability to yield median UMIs/cell as compared to a reaction comprising a reverse transcriptase having the amino acid sequence set forth in SEQ ID NO:1.

Embodiments of the application provide engineered fusion reverse transcriptases wherein the engineered reverse transcriptase has an amino acid sequence at least 95% identical to the amino acid sequence set forth in SEQ ID NO:1 and wherein the amino acid sequence of the engineered reverse transcriptase comprises at least one mutation indexed to SEQ ID NO:7 selected from the group consisting of a M17 mutation; an A32 mutation, a M44 mutation, a M39V mutation, a K47 mutation, a P51 mutation, an M66 mutation, an S67 mutation, an E69 mutation, a L72 mutation, a W94 mutation, a K103 mutation, an R110 mutation, a P117 mutation, an L139 mutation, an N178 mutation, an E179 mutation, a T197 mutation, a D200 mutation, an E201 mutation, an H204 mutation, a Q221 mutation, a V223 mutation, a V238 mutation, a G248 mutation, a T265 mutation, an E268 mutation, an R279 mutation, an R280 mutation, a K284 mutation, a T287 mutation, a F291 mutation, an E302 mutation, a T306 mutation, a P308 mutation, an F309 mutation, a W313 mutation, a T330 mutation, a Y344 mutation, an 1347 mutation, a C387 mutation, a W388 mutation, an R389 mutation, a C409 mutation, an R411 mutation, a G413 mutation, an A426 mutation, a G427 mutation, an L435G mutation, an L435K mutation, a P448 mutation, a D449G mutation, an R450 mutation, an N454 mutation, an A480 mutation, an H481 mutation, a N502 mutation, an A502 mutation, an H503 mutation, a D524N mutation, an H572 mutation, a W581 mutation, a D583 mutation, a K585 mutation, an H594 mutation, an L603 mutation, an H612 mutation, a P614 mutation, a G615 mutation, an H634 mutation, a P636 mutation, and a G637 mutation.

In various aspects the engineered reverse transcriptase has an amino acid sequence at least 95% identical to the amino acid sequence set forth in SEQ ID NO:1, and wherein the amino acid sequence of the engineered reverse transcriptase comprises a combination of mutations indexed to SEQ ID NO:7 selected from the group consisting of (i) an E69K mutation, an E302R mutation, a T306K mutation, a W313F mutation, an L435G mutation, and an N454K mutation, and further comprising at least one mutation selected from the group consisting of an M39V mutation, an M66L mutation, an L139P mutation, an F155Y mutation, a D200N mutation, an E201Q mutation, a T287A mutation, a T330P mutation, an R411F mutation, a P448A mutation, an H503V mutation, an H594K mutation, L603W mutation, an E607K mutation, an H634Y mutation, a G637R mutation and an H638G mutation; (ii) an L139P mutation, a D200N mutation, a T330P mutation, an L603W mutation, and an E607K mutation, and further comprising at least one mutation selected from the group consisting of: an M39V mutation, an M66L mutation, an E69K mutation, an F155Y mutation, an E201Q mutation, a T287A mutation, an E302R mutation, a T306K mutation, a W313F mutation, an R411F mutation, an L435G mutation, a P448A mutation, a D449G mutation, an N454K mutation, an H503V mutation, an H594K mutation, an H634Y mutation, a G637R mutation and an H638G mutation; (iii) an A32V mutation, an L72R mutation, a D200C mutation, a G248C mutation, an E286R mutation, an E302R mutation, a W388R mutation, and an L435G mutation; and (iv) a Y344L mutation and an I347L mutation.

Methods of performing a reverse transcription reaction for generating a nucleic acid product from an RNA template using an engineered fusion reverse transcriptase from any of the claims. In an aspect of the methods, the engineered fusion reverse transcriptase is a transcriptase comprising: at least one DNA binding domain selected from the group of DNA binding domains comprising archaeal DNA binding domains and single-stranded DNA binding domains and an engineered reverse transcriptase having an amino acid sequence that is at least 90% identical to SEQ ID NO:1, wherein said engineered reverse transcriptase comprises an M39 mutation, a K47 mutation, an L435 mutation, a D449 mutation, a D524 mutation, an E607 mutation, a D653 mutation and an L671 mutation as indexed to SEQ ID NO:7. In an aspect of the methods, the engineered fusion reverse transcriptase wherein the amino acid sequence of said DNA binding domain has been altered to reduce RNAase activity and further wherein the alteration to the amino acid sequence of said DNA binding domain is selected from the group comprising a K13 mutation, a K13L mutation, a D36 mutation, and a D36L mutation. In aspects of the methods, the amino acid sequence of said engineered reverse transcriptase comprises an amino acid sequence selected from the group of amino acid sequences set forth in SEQ ID NO:3, SEQ ID NO:5, SEQ ID NO:6, and SEQ ID NO:8.

In aspects of the methods, the amino acid sequence of the engineered fusion reverse transcriptase further comprises a second combination of mutations indexed to SEQ ID NO:7 consisting of: an E69K mutation, an E302R mutation, a T306K mutation, a W313F mutation, an L435G mutation, and an N454K mutation, and further comprising at least one mutation selected from the group consisting of an M39V mutation, an M66L mutation, an L139P mutation, an F155Y mutation, a D200N mutation, an E201Q mutation, a T287A mutation, a T330P mutation, an R411F mutation, a P448A mutation, a D449G mutation, an H503V mutation, an H594K mutation, L603W mutation, an E607K mutation, an H634Y mutation, a G637R mutation and an H638G mutation. In aspects of the methods, the amino acid sequence of said engineered fusion reverse transcriptase further comprises a second combination of mutations indexed to SEQ ID NO:7 consisting of: an L139P mutation, a D200N mutation, a T330P mutation, an L603W mutation, and an E607K mutation, and further comprising at least one mutation selected from the group consisting of: an M39V mutation, an M66L mutation, an E69K mutation, an F155Y mutation, an E201Q mutation, a T287A mutation, an E302R mutation, a T306K mutation, a W313F mutation, an R411F mutation, an L435G mutation, a P448A mutation, a D449G mutation, an N454K mutation, an H503V mutation, an H594K mutation, an H634Y mutation, a G637R mutation and an H638G mutation. In aspects of the methods, the amino acid sequence of said engineered reverse transcriptase further comprises a second combination of mutations indexed to SEQ ID NO:7 consisting of: an A32V mutation, an L72R mutation, a D200C mutation, a G248C mutation, an E286R mutation, an E302R mutation, a W388R mutation, and an L435G mutation. In aspects of the methods, the amino acid sequence of said engineered reverse transcriptase further comprises a second combination of mutations indexed to SEQ ID NO:7 consisting of: a Y344L mutation and an I347L mutation.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1 provides a schematic of an exemplary assay process. 5′-end labeled DNA primers are hybridized to RNA templates at room temperature (approx. 25° C.). Poly rG-labeled template switching oligonucleotides (rG-TSO) are added to the reaction mixture. The temperature is raised to 53° C.; first strand cDNA synthesis, the addition of a poly-C tail (tailing), template switching and TSO extension occur. Samples are transferred to a Genetic Analyzer for analysis.

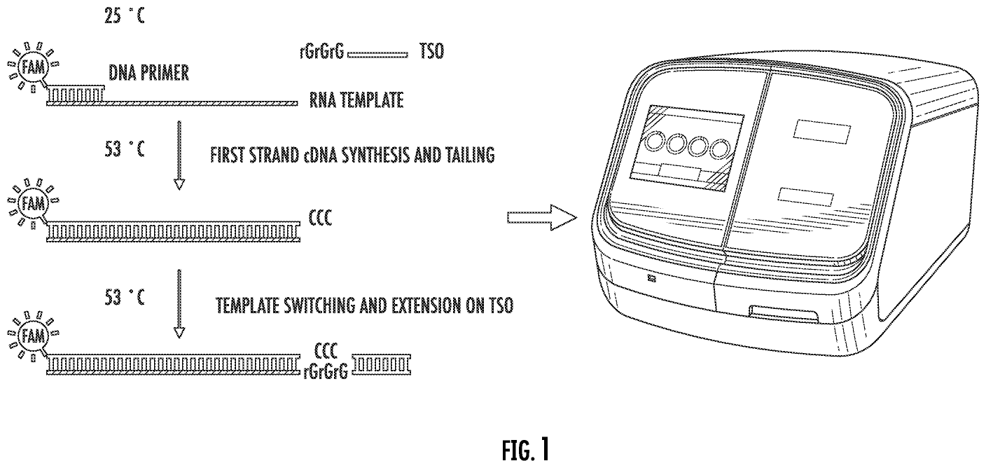

FIG. 2 provides an exemplary trace of an assay output following the process from FIG. 1. Product size was calibrated with synthetically sized controls for the primer alone size, a full-length extension of the primer length, and a full-length extension of the primer plus TSO. Product length is indicated on the x-axis, fluorescent signal intensity is indicated on the y-axis.

FIG. 3 provides an exemplary trace of capillary electrophoresis (CE) an assay output for an RT enzyme control (enzyme mix C, bottom) and an engineered reverse transcriptase having the amino acid sequence set forth in SEQ ID NO:14, top. See for example PCT/US20/64323 regarding the engineered reverse transcriptase having the amino acid sequence set forth in SEQ ID NO:14. Product length is indicated on the x-axis; fluorescent signal intensity is indicated on the y-axis. Peaks associated with the full-length product, the full-length product plus tail and the full-length product plus tail and template switching are indicated. The trace indicates the control RT reaction (enzyme mix C) yields full sized template switched products. The trace indicates reactions with an engineered reverse transcriptase enzyme having the amino acid sequence set forth in SEQ ID NO:14 yield full length transcription products, however a full-length template switched product peak is not significantly present.

FIG. 4 provides an exemplary trace of assay output for control enzyme mix C and the length parameters associated with various reaction products as used for transcription efficiency and template switching efficiency calculations. Reads less than 45 nucleotides are considered incomplete (section 1). Reads including the full length and the full length plus the tail are considered the elongation and tailing phase (section 2). Reads longer than the full length plus the tail and shorter than the full length plus tail and template switching are considered incomplete template switching products (incomplete TSO, section 3). Reads having the full length plus tail and template switching length are considered template switched (TSO, section 4). Transcription efficiency is the sum of the area under the curve for section 2, section 3 and section 4 divided by the total area under the curve. Template switching efficiency is the area under the curve of the template switched (section 4) divided by the sum of the area under curve for section 2, section 3 and section 4.

FIG. 5 provides a chart summarizing the percent of valid barcodes (y axis) in reads obtained for a control Enzyme Mix C, a reverse transcriptase having the amino acid sequence set forth in SEQ ID NO:1, an engineered reverse transcriptase having the amino acid sequence set forth in SEQ ID NO:6 and an engineered reverse transcriptase having the amino acid sequence set forth in SEQ ID NO:8, as assayed using a GEM-X assay.

FIG. 6 provides a chart summarizing the percent of reads confidently mapped to the transcriptome (y axis) obtained for a control Enzyme Mix C, a reverse transcriptase having the amino acid sequence set forth in SEQ ID NO:1, an engineered reverse transcriptase having the amino acid sequence set forth in SEQ ID NO:6, and an engineered reverse transcriptase having the amino acid sequence set forth in SEQ ID NO:8 as assayed using a GEM-X assay.

FIG. 7 provides a chart summarizing the median genes per cell (y axis) obtained for a control Enzyme Mix C, a reverse transcriptase having the amino acid sequence set forth in SEQ ID NO:1, an engineered reverse transcriptase having the amino acid sequence set forth in SEQ ID NO:6, and an engineered reverse transcriptase having the amino acid sequence set forth in SEQ ID NO:8 as assayed using a GEM-X assay.

FIG. 8 provides a chart summarizing the median UMI counts per cell (y axis) obtained for a control Enzyme Mix C, a reverse transcriptase having the amino acid sequence set forth in SEQ ID NO:1, an engineered reverse transcriptase having the amino acid sequence set forth in SEQ ID NO:6, and an engineered reverse transcriptase having the amino acid sequence set forth in SEQ ID NO:8 as assayed using a GEM-X assay.

FIG. 9 provides a chart summarizing the fraction of ribosomal protein UMI counts per cell (y axis) Enzyme Mix C, a reverse transcriptase having the amino acid sequence set forth in SEQ ID NO:1, an engineered reverse transcriptase having the amino acid sequence set forth in SEQ ID NO:6, and an engineered reverse transcriptase having the amino acid sequence set forth in SEQ ID NO:8 as assayed using a GEM-X assay.

FIG. 10 provides a chart summarizing the fraction of mitochondrial UMI counts per cell (y axis) obtained for a control Enzyme Mix C, a reverse transcriptase having the amino acid sequence set forth in SEQ ID NO:1, an engineered reverse transcriptase having the amino acid sequence set forth in SEQ ID NO:6, and an engineered reverse transcriptase having the amino acid sequence set forth in SEQ ID NO:8 as assayed using a GEM-X assay.

FIG. 11 provides a summary of results obtained when assessing a variety of engineered reverse transcriptases for transcription efficiency and template switching efficiency. The template switching efficiency of a fusion variant having the amino acid sequence set forth in SEQ ID NO:8 is greater than the template switching efficiency of enzymes having an amino acid sequence set forth in SEQ ID NO:1 or SEQ ID NO:6. Y-axis is the % of generated nucleic acid product.

FIG. 12 provides a summary of results obtained from an experiment evaluating template switching ability of an enzyme having the amino acid sequence set forth in SEQ ID NO:1 and an engineered reverse transcriptase having the amino acid sequence set forth in SEQ ID NO: 5. The template switching efficiency of the engineered reverse transcriptase having the amino acid sequence set forth in SEQ ID NO:5 is significantly increased compared to the template switching efficiency of an engineered reverse transcriptase having the amino acid sequence set forth in SEQ ID NO:1.

### DETAILED DESCRIPTION

In embodiments of the present disclosure, “engineered fusion reverse transcriptases” and “engineered fusion reverse transcription enzymes” comprise at least one DNA binding domain and an engineered reverse transcriptase. The DNA binding domain and the engineered reverse transcriptase portions of an engineered fusion reverse transcriptase may be immediately adjacent to each other or separated by a linker region. The DNA binding domain may be selected from the group of DNA binding domains comprising archaeal DNA binding domains and single-stranded DNA binding domains. A DNA binding domain may be N-terminal to the engineered reverse transcriptase, C-terminal to the engineered reverse transcriptase, at the C-terminus of the engineered fusion reverse transcriptase, or at the N-terminus of the engineered fusion reverse transcriptase. When the engineered fusion reverse transcriptase comprises at least two DNA binding domains, the DNA binding domains may be at the same terminus or at different termini. The at least two DNA binding domains may be at least two of the same DNA binding domains or at least two different DNA binding domains.

DNA binding domain (DBD) proteins or polypeptides are capable of binding DNA. DNA binding domains may include, but are not limited to, archaeal DNA binding domains, single-stranded DNA binding domains and 7 kDa DNA binding domains. Archaeal DNA binding domains are obtained from archaebacterial proteins and may include, but are not limited to, Sto7, Sso7d, Sis7b, Sis7a, Ssh7b, Sto7, Aho7C, Aho7B, Aho7A, Mcu7, Mse7, Sac7e, and Sac7d. An archaeal DNA binding domain may comprise an archaeal DNA binding domain consensus motif having the amino acid sequence set forth in SEQ ID NO:2. Sto7 is a DBD from Sulfolobus tokadaii; the Sto7 amino acid sequence is set forth in SEQ ID NO:12. 7 kDa DBD's may include, but are not limited to, DBDs approximately 7 kDa, Sto7 and Sso7d. Sso7d is a DBD from Sulfolobus solfataricus; the Sso7d amino acid sequence is set forth in SEQ ID NO:13. Single-stranded DNA binding domains preferentially bind single-stranded DNA. DBD's may comprise one or more site specific alterations including, but not limited to a K13 alteration, such as a K13L alteration, wherein such alterations may alter one or more aspects of DNA binding. The alteration may be an increase or decrease in an aspect of DNA binding. Furthermore, it is recognized that an alteration that increases one aspect of DNA binding may alter a different aspect of DNA binding; the alteration of a different aspect of DNA binding may be an increase or a decrease.

Reverse transcriptases or reverse transcription enzymes are known in the art; reverse transcriptases perform a reverse transcription reaction. “Reverse transcriptase” and “reverse transcription enzyme” are synonymous. In some embodiments, reverse transcription is initiated by hybridization of a priming sequence to an RNA molecule which is extended by an engineered reverse transcription enzyme in a template directed fashion. In some embodiments, a reverse transcription enzyme adds a plurality of non-template oligonucleotides to a nucleotide strand. In some embodiments, the reverse transcription reaction produces single stranded complementary deoxyribonucleic acid (cDNA) molecules each having a molecular tag on a 5′ end thereof, followed by amplification of cDNA to produce a double stranded DNA having the molecular tag on the 5′ end and a 3′ end of the double stranded DNA. As used herein, the term “wild-type” refers to a gene or gene product that has the characteristics of that gene or gene product when isolated from a naturally occurring source. The amino acid sequence set forth in SEQ ID NO:7 is a wild-type MMLV amino acid sequence.

An engineered fusion reverse transcriptase may exhibit one or more reverse transcriptase related activities including but not limited to, RNA-dependent DNA polymerase activity, RNAse H activity, DNA-dependent DNA polymerase activity, RNA binding activity, DNA binding activity, polymerase activity, primer extension activity, strand-displacement activity, helicase activity, strand transfer activity, template binding activity, transcription template switching, transcription efficiencies, template switching efficiencies, processivity efficiencies, incorporation efficiencies, fidelity efficiencies, polymerization efficiencies, altered specificity, altered non-templated base addition, altered thermostability, altered tailing, altered adapter binding, binding efficiencies, and altered binding affinities. It is recognized that a change in any activity may increase, decrease or have no effect on a different reverse-transcriptase related activity. It is also recognized that a change in one activity may alter multiple properties of a reverse transcriptase. It is understood that when multiple properties are affected, the properties may be altered similarly or differently. It is further recognized that methods of evaluating reverse transcriptase related activities are known in the art. Change in a reverse transcriptase related activity may alter one or more of the following results including but not limited to the yield of unique molecular identifiers (UMI), the median UMI obtained, the yield of mitochondrial UMI counts, and the yield of ribosomal UMI counts. A change or alteration in the yield of unique molecular identifiers (UMI) the median UMI obtained, the yield of mitochondrial UMI counts, and/or the yield of ribosomal UMI counts may indicate one or more altered reverse transcriptase related activities.

In some embodiments, the fusion domain may occur at the N-terminus or C-terminus of the variant engineered reverse transcriptase amino acid sequence. Further, an engineered reverse transcription enzyme may comprise a DBD fusion domain at the N-terminus and C-terminus of the reverse transcriptase amino acid sequence. In some embodiments, a DBD fusion domain occurs at the actual N-terminus or C-terminus of the entire polypeptide. In some embodiments, a DBD fusion domain occurs at the N-terminus or C-terminus of the engineered reverse transcriptase amino acid sequence and is internal to an additional affinity tag. The amino acid sequence of a DNA binding domain consensus motif is set forth in SEQ ID NO:2.

DNA binding involves multiple aspects or properties related to an enzyme's ability to interact with and bind to a DNA molecule. DNA binding related properties may include, but are not limited to, processivity, clamping, off rate and on rate kinetics, template switching and RNase activity.

In various embodiments, the amino acid sequence of the engineered reverse transcriptase comprises a Sto7 DNA binding domain at the C-terminus. In various embodiments, the amino acid sequence of the engineered reverse transcriptase comprises an Ss07d DNA binding domain at the N-terminus or an Ss07d DNA binding domain at the C-terminus, or vice versa.

In some embodiments, engineered reverse transcription enzymes may further comprise an affinity tag at the N-terminus or at a C-terminus of the amino acid sequence. In some instances, the affinity tag may include, but is not limited to, albumin binding protein (ABP), AU1 epitope, AU5 epitope, T7-tag, V5-tag, B-tag, Chloramphenicol Acetyl Transferase (CAT), Dihydrofolate reductase (DHFR), AviTag, Calmodulin-tag, polyglutamate tag, E-tag, FLAG-tag, HA-tag, Myc-tag, NE-tag, S-tag, SBP-tag, Doftag 1, Softag 3, Spot-tag, tetracysteine (TC) tag, Ty tag, VSV-tag, Xpress tag, biotin carboxyl carrier protein (BCCP), green fluorescent protein tag, HaloTag, Nus-tag, thioredoxin-tag, Fc-tag, cellulose binding domain, chitin binding protein (CBP), choline-binding domain, galactose binding domain, maltose binding protein (MBP), Horseradish Peroxidase (HRP), Strep-tag, HSV epitope, Ketosteroid isomerase (KSI), KT3 epitope, LacZ, Luciferase, PDZ domain, PDZ ligand, Polyarginine (Arg-tag), Polyaspartate (Asp-tag), Polycysteine (Cys-tag), Polyphenylalanine (Phe-tag), Profinity eXact, Protein C, S1-tag, S1-tag, Staphylococcal protein A (Protein A), Staphylococcal protein G (Protein G), Small Ubiquitin-like Modifier (SUMO), Tandem Affinity Purification (TAP), TrpE, Ubiquitin, Universal, glutathione-S-transferase (GST), and poly(His) tag.

In some embodiments, an engineered reverse transcription enzyme further comprises a protease cleavage sequence, wherein cleavage by a protease results in cleavage of the affinity tag from the engineered reverse transcription enzyme. In some instances, the protease cleavage sequence is recognized by a protease including, but not limited to, alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase. In some instances, the protease cleavage sequence is a thrombin cleavage sequence.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention which can be had by reference to the specification as a whole. Accordingly, the terms defined immediately below are more fully defined by reference to the specification as a whole.

As used herein, “purified” means that a molecule is present in a sample at a concentration of at least 95% by weight, or at least 98% by weight of the sample in which it is contained.

The term “% homology” is used interchangeably herein with the term “% identity” herein and refers to the level of nucleic acid or amino acid sequence identity between the nucleic acid sequence that encodes any one of the inventive reverse transcriptases or the inventive reverse transcriptase's amino acid sequence, when aligned using a sequence alignment program.

“Variant” means a protein which is derived from a precursor protein (such as the native protein, for example MMLV native protein as set forth in SEQ ID NO:7) by addition of one or more amino acids to either or both the C- and N-terminal end, substitution of one or more amino acids at one or a number of different sites in the amino acid sequence, deletion of one or more amino acids at either or both ends of the protein or at one or more sites in the amino acid sequence, or addition of a fusion domain. SEQ ID NO:1 is a variant of MMLV. The preparation of an enzyme variant is preferably achieved by modifying a DNA sequence which encodes for the wild-type protein, transformation of that DNA sequence into a suitable host, and expression of the modified DNA sequence to form the derivative enzyme. A variant reverse transcriptase of the invention includes a protein comprising altered amino acid sequences in comparison with a precursor enzyme amino acid sequence wherein the variant reverse transcriptase retains the characteristic enzymatic nature of the precursor enzyme but which may have altered properties in some specific aspect. For example, an engineered reverse transcriptase variant may have an altered pH optimum or increased temperature stability but may retain its characteristic transcriptase activity. A “variant” may have at least about 45%, at least about 50%, at least about 55%, at least about 60%, at least about 65%, at least about 70%, at least about 75%, at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, or at least about 99.5% sequence identity to a polypeptide sequence when optimally aligned for comparison. Percent identity may pertain to the percent identity of the DNA binding domain or the engineered reverse transcriptase portion of an engineered fusion reverse transcriptase. As used herein, a variant residue position is described in relation to the wild-type or precursor amino acid sequence set forth in SEQ ID NO:7; the amino acid position is indexed to SEQ ID NO:7. A fusion variant further comprises at least one fusion domain selected from the group of DNA binding domains described elsewhere herein.

As used herein, a protein having a certain percent (e.g., at least 90%, at least 91%, at least 92%, at least 93%, at least 94%, at least 95%, at least 96%, at least 97%, at least 98%, or at least 99%) of sequence identity with another sequence means that, when aligned, that percentage of bases or amino acid residues are the same in comparing the two sequences. This alignment and the percent homology or identity can be determined using any suitable software program known in the art, for example those described in CURRENT PROTOCOLS IN MOLECULAR BIOLOGY, Ausubel et al., eds., 1987, Supplement 30, section 7.7.18. Representative programs include the Vector NTI Advance™ 9.0 (Invitrogen Corp. Carlsbad, CA), GCG Pileup, FASTA (Pearson et al. (1988) Proc. Natl Acad. ScL USA 85:2444-2448), and BLAST (BLAST Manual, Altschul et al., Nat'l Cent. Biotechnol. Inf., Nat'l Lib. Med. (NCIB NLM NIH), Bethesda, Md., and Altschul et al., (1997) Nucleic Acids Res. 25:3389-3402) programs. Another typical alignment program is ALIGN Plus (Scientific and Educational Software, PA), generally using default parameters. Other sequence alignment software programs that find use are the TFASTA Data Searching Program available in the Sequence Software Package Version 6.0 (Genetics Computer Group, University of Wisconsin, Madison, WI and CLC Main Workbench (Qiagen) Version 20.0. The present disclosure is not limited to the software being used to align two or more sequences.

In some embodiments, the engineered fusion reverse transcription enzyme comprises at least one DNA binding domain selected from the group of DNA binding domains comprising archaeal DNA binding domains and single-stranded DNA binding domains and an amino acid sequence that is at least 90% identical to a reverse transcriptase having the amino acid sequence set forth in SEQ ID NO:1. In other embodiments, the engineered reverse transcriptase exhibits an altered reverse transcriptase activity as compared to a reverse transcriptase having the amino acid sequence set forth in SEQ ID NO:1.

In some embodiments, the engineered reverse transcription enzyme comprises an amino acid sequence that is at least 95% identical to SEQ ID NO: 1 and wherein the amino acid sequence of the engineered reverse transcriptase comprises at least one mutation indexed to SEQ ID NO:7 selected from the group comprising, or consisting essentially of a M17 mutation; an A32 mutation, a M44 mutation, a M39 mutation, a K47 mutation, a P51 mutation, an M66 mutation, an S67 mutation, an E69 mutation, a L72 mutation, a W94 mutation, a K103 mutation, an R110 mutation, a P117 mutation, an L139 mutation, an F155 mutation, an N178 mutation, an E179 mutation, a T197 mutation, a D200 mutation, an E201 mutation, an H204 mutation, a Q221 mutation, a V223 mutation, a V238 mutation, a G248 mutation, a T265 mutation, an E268 mutation, an R279 mutation, an R280 mutation, a K284 mutation, a T287 mutation, a F291 mutation, an E302 mutation, an E302K mutation, an E302R mutation, a T306 mutation, a T306R mutation, a T306K mutation a P308 mutation, an F309 mutation, a W313 mutation, a T330 mutation, a Y344 mutation, an 1347 mutation, a C387 mutation, a W388 mutation, an R389 mutation, a C409 mutation, an R411 mutation, a G413 mutation, an A426 mutation, a G427 mutation, an L435 mutation, an L435G mutation, an L435K mutation, a P448 mutation, a D449 mutation, an R450 mutation, a n N454 mutation, an A480 mutation, an H481 mutation, a N502 mutation, an A502 mutation, an H503 mutation, a D524 mutation, an H572 mutation, a W581 mutation, a D583 mutation, a K585 mutation, an H594 mutation, an L603 mutation, an E607 mutation, an H612 mutation, a P614 mutation, a G615 mutation, an H634 mutation, a P636 mutation, a G637 mutation, an H638 mutation, a D653 mutation, and an L671 mutation, further including a DBD sequence. In some embodiments, the engineered reverse transcription enzyme comprises an amino acid sequence that is at least 95% identical to SEQ ID NO: 1 and wherein the amino acid sequence of the engineered reverse transcriptase comprises an M39 mutation, a K47 mutation, an L435 mutation, a D449 mutation, a D524 mutation, an E607 mutation, a D653 mutation, and an L671 mutation as indexed to SEQ ID NO:7 and further comprising at least one mutation indexed to SEQ ID NO:7 selected from the group comprising, or consisting essentially of a M17 mutation; an A32 mutation, a M44 mutation, a M39V mutation, a P51 mutation, an M66 mutation, an S67 mutation, an E69 mutation, a L72 mutation, a W94 mutation, a K103 mutation, an R110 mutation, a P117 mutation, an L139 mutation, an F155 mutation, an N178 mutation, an E179 mutation, a T197 mutation, a D200 mutation, an E201 mutation, an H204 mutation, a Q221 mutation, a V223 mutation, a V238 mutation, a G248 mutation, a T265 mutation, an E268 mutation, an R279 mutation, an R280 mutation, a K284 mutation, a T287 mutation, a F291 mutation, an E302 mutation, an E302K mutation, an E302R mutation, a T306 mutation, a T306R mutation, a T306K mutation a P308 mutation, an F309 mutation, a W313 mutation, a T330 mutation, a Y344 mutation, an 1347 mutation, a C387 mutation, a W388 mutation, an R389 mutation, a C409 mutation, an R411 mutation, a G413 mutation, an A426 mutation, a G427 mutation, an L435G mutation, an L435K mutation, a P448 mutation, a D449G mutation, an R450 mutation, a n N454 mutation, an A480 mutation, an H481 mutation, a N502 mutation, an A502 mutation, an H503 mutation, a D524N mutation, an H572 mutation, a W581 mutation, a D583 mutation, a K585 mutation, an H594 mutation, an L603 mutation, an H612 mutation, a P614 mutation, a G615 mutation, an H634 mutation, a P636 mutation, a G637 mutation, and an H638 mutation, further including a DBD sequence. In other embodiments, the engineered fusion reverse transcription enzyme exhibits an altered reverse transcriptase related activity when compared to a reverse transcriptase having the amino acid sequence set forth in SEQ ID NO:1.

In some embodiments, an engineered reverse transcriptase comprises an amino acid sequence that is at least 95% identical to SEQ ID NO:1. In other embodiments, the engineered reverse transcriptase exhibits an altered reverse transcriptase related activity as compared to a reverse transcriptase having the amino acid sequence set forth in SEQ ID NO:1. In additional embodiments, the engineered reverse transcriptase comprises a combination of mutations indexed to SEQ ID NO:7 selected from the group consisting of i) an E69K mutation, an E302R mutation, a T306K mutation, a W313F mutation, a L435G mutation, and an N454K mutation, and further comprising at least one mutation selected from the group consisting of an M39V mutation, an M66L mutation, an L139P mutation, an F155Y mutation, a D200N mutation, an E201Q mutation, a T287A mutation, a T330P mutation, an R411F mutation, a P448A mutation, a D449G mutation, an H503V mutation, an H594K mutation, L603W mutation, an E607K mutation, an H634Y mutation, a G637R mutation and an H638G mutation; ii) an L139P mutation, a D200N mutation, a T330P mutation, an L603W mutation, and an E607K mutation, and further comprising at least one mutation selected from the group consisting of: an M39V mutation, an M66L mutation an E69K mutation, an F155Y mutation, an E201Q mutation, a T287A mutation, an E302R mutation, a T306K mutation, a W313F mutation, an R411F mutation, an L435G mutation, a P448A mutation, a D449G mutation, an N454K mutation, an H503V mutation, an H594K mutation, an H634Y mutation, a G637R mutation and an H638G mutation; iii) an A32V mutation, an L72R mutation, a D200C mutation, a G248C mutation, an E286R mutation, an E302R mutation, a W388R mutation, and an L435G mutation; and iv) a Y344L mutation and an I347L mutation. A variant may comprise a first combination of mutations or alterations and may further comprise an additional or second combination of mutations. A first combination of mutations or alterations may include, but is not limited to, a combination set forth herein: a M39 mutation, a K47 mutation, an L435 mutation, a D449 mutation, a D524 mutation, an E607 mutation, a D653 mutation and an L671 mutation; an M39V mutation, a K47 mutation, an L435K mutation, a D449G mutation, a D524N mutation, an E607 mutation, a D653 mutation and an L671 mutation; an M39 mutation, an M66 mutation, an E302 mutation, a T306 mutation, an L435 mutation, a D449 mutation, a D524 mutation, an E607 mutation, a D653 mutation and an L671 mutation; an M39 mutation, an M66 mutation, an E302 (K or R) mutation, a T306 (R or K) mutation, an L435 (K or G), a D449 mutation, a D524 mutation, an E607 (G or K) mutation, a D653 mutation, and an L671 mutation; and an M39V mutation, an M66 mutation, an E302 (K or R) mutation, a T306 (R or K) mutation, an L435 (K or G), a D449G mutation, a D524N mutation, an E607 (G or K) mutation, a D653 mutation, and an L671 mutation.

The second combination of mutations in a first engineered reverse transcriptase may comprise either a totally different set of mutations or a partially different second set of mutations as in a second engineered reverse transcriptase. A second combination of mutations or alterations may include but is not limited to (a) one or more mutations selected from the group comprising an M17 mutation; an A32 mutation, a M44 mutation, a P51 mutation, an M66 mutation, an S67 mutation, an E69 mutation, a L72 mutation, a W94 mutation, a K103 mutation, an R110 mutation, a P117 mutation, an L139 mutation, an F155 mutation, an N178 mutation, an E179 mutation, a T197 mutation, a D200 mutation, an E201 mutation, an H204 mutation, a Q221 mutation, a V223 mutation, a V238 mutation, a G248 mutation, a T265 mutation, an E268 mutation, an R279 mutation, an R280 mutation, a K284 mutation, a T287 mutation, a F291 mutation, an E302 mutation, an E302K mutation, an E302R mutation, a T306 mutation, a T306R mutation, a T306K mutation, a P308 mutation, an F309 mutation, a W313 mutation, a T330 mutation, a Y344 mutation, an 1347 mutation, a C387 mutation, a W388 mutation, an R389 mutation, a C409 mutation, an R411 mutation, a G413 mutation, an A426 mutation, a G427 mutation, an L435G mutation, an L435K mutation, a P448 mutation, a D449G mutation, an R450 mutation, an N454 mutation, an A480 mutation, an H481 mutation, a N502 mutation, an A502 mutation, an H503 mutation, a D524N mutation, an H572 mutation, a W581 mutation, a D583 mutation, a K585 mutation, an H594 mutation, an L603 mutation, an H612 mutation, a P614 mutation, a G615 mutation, an H634 mutation, a P636 mutation, a G637 mutation, and an H638 mutation; (b) an E69K mutation, an E302R mutation, a T306K mutation, a W313F mutation, an L435G mutation, and an N454K mutation, and further comprising at least one mutation selected from the group consisting of an M39V mutation, an M66L mutation, an L139P mutation, an F155Y mutation, a D200N mutation, an E201Q mutation, a T287A mutation, a T330P mutation, an R411F mutation, a P448A mutation, a D449G mutation, an H503V mutation, an H594K mutation, L603W mutation, an E607K mutation, an H634Y mutation, a G637R mutation and an H638G mutation; (c) an L139P mutation, a D200N mutation, a T330P mutation, an L603W mutation, and an E607K mutation, and further comprising at least one mutation selected from the group consisting of: an M39V mutation, an M66L mutation, an E69K mutation, an F155Y mutation, an E201Q mutation, a T287A mutation, an E302R mutation, a T306K mutation, a W313F mutation, an R411F mutation, an L435G mutation, a P448A mutation, a D449G mutation, an N454K mutation, an H503V mutation, an H594K mutation, an H634Y mutation, a G637R mutation and an H638G mutation; (d) an A32V mutation, an L72R mutation, a D200C mutation, a G248C mutation, an E286R mutation, an E302R mutation, a W388R mutation, and an L435G mutation; and (e) a Y344L mutation and an I347L mutation. It is recognized that the second combination of mutations may comprise a group of mutations as described herein and one or more additional mutations.

In some embodiments, the engineered reverse transcription enzyme is engineered to have reduced and/or abolished RNase activity. In some embodiments, the engineered reverse transcription enzyme is engineered to have reduced and/or abolished RNase H activity. In some embodiments, the engineered reverse transcription enzyme engineered to have reduced and/or abolished RNase H activity comprises a D524 mutation.

In some embodiments, the DNA binding domain fusion exhibits reduced RNAase activity. In some embodiments, the amino acid sequence of the DNA binding domain has been altered to reduce RNAase activity. In some aspects, the amino acid sequence of the DNA binding domain portion of the fusion polypeptide has an alteration that impacts RNAase activity. Alterations to the amino acid sequence that may alter RNAase activity include, but are not limited to, a K13 mutation, a K13L mutation, a D36 mutation, and a D36L mutation. The amino acid sequence of an engineered fusion reverse transcriptase comprises a Sto7 DNA binding domain at the C-terminus of the polypeptide, wherein the DNA binding domain comprises a K13 mutation as provided in SEQ ID NO:3.

The engineered fusion reverse transcription enzyme variants of the present disclosure unexpectedly provided an altered reverse transcriptase activity, such as but not limited to, improved processivity, template switching efficiency, chemical tolerance, thermal stability, processive reverse transcription, non-templated base addition, and template switching ability. An engineered reverse transcription enzyme of the current application may exhibit an altered base-biased template switching activity such as an increased base-biased template switching activity, decreased base-biased template switching activity or an altered base-bias to the template switching activity. An engineered reverse transcriptase variant may exhibit enhanced template switching with a 5′-G cap on the nucleic acid. Furthermore, engineered reverse transcription enzyme variants described herein may also exhibit unexpectedly higher tolerance to inhibitory compositions which might be present in cell lysates (i.e., are less inhibited by cell lysates) than that exhibited by an enzyme having the amino acid sequence set forth in SEQ ID NO:1. Further, engineered reverse transcription enzyme variants of the present disclosure may have an unexpectedly greater ability to associate or bind to full-length transcripts (e.g., in T-cell receptor paired transcriptional profiling), as compared to that exhibited by an enzyme having the amino acid sequence set forth in SEQ ID NO:1. It is recognized that salt concentration, the concentration of a cell fixation chemical and/or the concentration of a process reagent in a reverse transcriptase reaction may impact function of a reverse transcriptase. For example, “chemical tolerance” is intended that an engineered fusion reverse transcription enzyme of the current application may exhibit a reverse transcriptase related activity in either an expanded salt concentration range or in the presence of an increased concentration of a cell fixation chemical or process reagent, or in both an expanded salt concentration range and in the presence of an increased concentration of a cell fixation chemical or process reagent, as compared to the reverse transcriptase related activity of an enzyme having the amino acid sequence set forth in SEQ ID NO:1.

An altered template switching efficiency may be an increased template switching efficiency or a decreased template switching efficiency as compared to the template switching efficiency of a reverse transcriptase having the amino acid sequence set forth in SEQ ID NO:1. Altered template switching efficiency may be at least 0.1×, 0.2×, 0.3×, 0.4×, 0.5×, 0.6×, 0.7×, 0.8×, 0.9×, 1×, 1.5×, 2×, 2.5×, 3×, 3.5×, 4×, 4.5×, 5×, 5.5×, 6×, 6.5×, 7×, 7.5×, 8×, 8.5×, 9× or at least 10× greater than the template switching activity of a reverse transcriptase having the amino acid sequence set forth in SEQ ID NO:1. Altered template switching efficiency may range from 0.1× greater to 10× greater than the template switching activity of a reverse transcriptase having the amino acid sequence set forth in SEQ ID NO:1, from 0.25× greater to 7.5× greater than the template switching activity of a reverse transcriptase having the amino acid sequence set forth in SEQ ID NO:1, from 0.5× greater to 5× greater than the template switching activity of a reverse transcriptase having the amino acid sequence set forth in SEQ ID NO:1, or from 1× greater to 4× greater than the template switching activity of a reverse transcriptase having the amino acid sequence set forth in SEQ ID NO:1.

An altered transcription efficiency may be an increased transcription efficiency or a decreased transcription efficiency as compared to the transcription efficiency of a reverse transcriptase having the amino acid sequence set forth in SEQ ID NO:1. Altered transcription efficiency may be at least 0.1×, 0.2×, 0.3×, 0.4×, 0.5×, 0.6×, 0.7×, 0.8×, 0.9×, 1×, 1.5×, 2×, 2.5×, 3×, 3.5×, 4×, 4.5×, 5×, 5.5×, 6×, 6.5×, 7×, 7.5×, 8×, 8.5×, 9×, 10×, 15×, 20×, 25× or at least 30× greater than the transcription efficiency of a reverse transcriptase having the amino acid sequence set forth in SEQ ID NO:1.

Processivity relates to a reverse transcriptase's ability to remain associated with the template while incorporating nucleotides. Measurements of processivity may include but are not limited to the number of nucleotides incorporated in a single binding event of a reverse transcriptase molecule. Processivity also relates to the affinity of the enzyme for the substrate; thus, an enzyme with increased processivity may be more resistant to the presence of an inhibitor.

The engineered reverse transcriptases of the present application may be used in any application in which a reverse transcriptase with the indicated altered activity is desired. Methods of using reverse transcriptases are known in the art; one skilled in the art may select any of the engineered reverse transcriptases disclosed herein. In some embodiments, the reverse transcriptases of the present disclosure are used in reverse transcription reactions, such as RT-PCR, or other known reactions in the art where nucleic acids, for example RNA molecules, are reverse transcribed using a reverse transcriptase. In some embodiments, a reverse transcription reaction introduces a bar code. In some embodiments, the barcoding reaction is an enzymatic reaction. In some embodiments, the barcoding reaction is a reverse transcription amplification reaction that generates complementary deoxyribonucleic acid (cDNA) molecules upon reverse transcription of ribonucleic acid (RNA) molecules of the cell. In some embodiments, the RNA molecules are released from the cell. In some embodiments, the RNA molecules are released from the cell by lysing the cell. In some embodiments, the RNA molecules are released from the cell by permeabilizing the cell, or a tissue which comprises a plurality of the same and/or different cell types. In some embodiments, the RNA molecules are messenger RNA (mRNA).

In some embodiments, the molecular tags are coupled to priming sequences and the barcoding reaction is initiated by hybridization of the priming sequences to the RNA molecules. In some embodiments, each priming sequence comprises a random N-mer sequence. In some embodiments, the random N-mer sequence is complementary to a 3′ sequence of a ribonucleic acid molecule of said cell. In some embodiments, the random N-mer sequence comprises a poly-dT sequence having a length of at least 5 bases. In some embodiments, the random N-mer sequence comprises a poly-dT sequence having a length of at least 10 bases (SEQ ID NO:4). In some embodiments, the barcoding reaction is performed by extending the priming sequences in a template directed fashion using reagents for reverse transcription. In some embodiments, the reagents for reverse transcription comprise a reverse transcription enzyme, a buffer and a mixture of nucleotides. In some embodiments, the reverse transcription enzyme adds a plurality of non-template oligonucleotides upon reverse transcription of a ribonucleic acid molecule. In some embodiments, the reverse transcription enzyme is an engineered fusion reverse transcription enzyme as disclosed herein.

In some embodiments, the barcoding reaction produces single stranded complementary deoxyribonucleic acid (cDNA) molecules each having a molecular tag on a 5′ end thereof, followed by amplification of the cDNA to produce a double stranded DNA having the molecular tag on the 5′ end and a 3′ end of the double stranded DNA.

In some embodiments, the molecular tags (e.g., barcode oligonucleotides) include unique molecular identifiers (UMIs). In some embodiments, the UMIs are oligonucleotides. In some embodiments, the molecular tags are coupled to priming sequences. In some embodiments, each of said priming sequences comprises a random N-mer sequence. In some embodiments, the random N-mer sequence is complementary to a 3′ sequence of said RNA molecules. In some embodiments, the priming sequence comprises a poly-dT sequence having a length of at least 5 bases. In some embodiments, the priming sequence comprises a poly-dT sequence having a length of at least 10 bases (SEQ ID NO:4). In some embodiments, the priming sequence comprises a poly-dT sequence having a length of at least 5 bases, at least 6 bases, at least 7 bases, at least 8 bases, at least 9 bases, at least 10 bases.

Unique molecular identifiers (UMIs), e.g., in the form of nucleic acid sequences are assigned or associated with individual cells or populations of cells, in order to tag or label the cell's components (and as a result, its characteristics). These unique molecular identifiers may be used to attribute the cell's components and characteristics to an individual cell or group of cells, additionally to be used as a method for counting the individual cells or groups of cells by their incorporation.

In some aspects, the unique molecular identifiers are provided in the form of nucleic acid molecules (e.g., oligonucleotides) that comprise nucleic acid barcode sequences that may be attached to or otherwise associated with the nucleic acid contents of individual cell, or to other components of the cell, and particularly to fragments of those nucleic acids. The nucleic acid molecule can, and do have differing barcode sequences, or at least represent a large number of different barcode sequences across all of the partitions in a given analysis. In some aspects only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

Moreover, when a population of barcodes is partitioned, the resulting population of partitions can also include a diverse barcode library that may include at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

The engineered reverse transcriptases of the present application may be suitable for use in methods in which a cell can be co-partitioned along with a nucleic acid barcode molecule bearing bead. The nucleic acid barcode molecules can be released from the bead in the partition. By way of example, in the context of analyzing sample RNA, the poly-dT (poly-deoxythymine, also referred to as oligo (dT)) segment of one of the released nucleic acid molecules can hybridize to the poly-A tail of a mRNA molecule. Reverse transcription results in a cDNA transcript of the mRNA, but which transcript includes each of the sequence segments of the nucleic acid molecule. Without being limited by mechanism, because the nucleic acid molecule comprises an anchoring sequence, it may be more likely hybridize to and prime reverse transcription at the sequence end of the poly-A tail of the mRNA. Within any given partition, all of the cDNA transcripts of the individual mRNA molecules may include a common barcode sequence segment. However, the transcripts made from the different mRNA molecules within a given partition may vary at the unique UMI segment. Beneficially, even following any subsequent amplification of the contents of a given partition, the number of different UMIs can be indicative of the quantity of mRNA originating from a given partition, and thus from the cell. As noted above, the transcripts can be amplified, cleaned up and sequenced to identify the sequence of the cDNA transcript of the mRNA, as well as to sequence the barcode segment and the UMI segment. While a poly-dT primer sequence is described, other targeted or random priming sequences may also be used in priming the reverse transcription reaction.

Template switching oligonucleotides (also referred to herein as “switch oligos” or “switch oligonucleotides”) may be used for template switching. In some cases, template switching can be used to increase the length, or help to secure the 5′ end, of a RNA transcript thereby helping to generate a full-length cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxylnosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination. Suitable lengths of a switch oligo are known in the art. See for example U.S. patent application Ser. No. 15/975,516, filed May 9, 2018, herein incorporated by reference in its entirety.

In various embodiments the poly-dT segment may be extended in a reverse transcription reaction using the mRNA as a template to produce a cDNA transcript complementary to the mRNA and also includes sequence segments of a barcode oligonucleotide. Terminal transferase activity of the reverse transcriptase can add additional bases to the cDNA transcript (e.g., polyC). The switch oligo may then hybridize with the additional bases added to the cDNA transcript and facilitate template switching. A sequence complementary to the switch oligo sequence can then be incorporated into the cDNA transcript via extension of the cDNA transcript using the switch oligo as a template. Within any given partition, all the cDNA transcripts of the individual mRNA molecules include a common barcode sequence segment. However, by including the unique random N-mer sequence, the transcripts made from different mRNA molecules within a given partition will vary at this unique sequence. As described elsewhere herein, this provides a quantification feature that can be identifiable even following any subsequent amplification of the contents of a given partition, e.g., the number of unique segments associated with a common barcode can be indicative of the quantity of mRNA originating from a single partition, and thus, a single cell. The cDNA transcript may then be amplified with PCR primers. The amplified product may then be purified (e.g., via solid phase reversible immobilization (SPRI)). The amplified product may be sheared, ligated to additional functional sequences, and further amplified (e.g., via PCR).

It is recognized that certain reverse transcriptase enzymes may increase UMI reads from genes of a desired length or length of interest due to the engineered reverse transcriptase's enhanced efficiencies. The desired length of genes may be selected from the group of lengths of less than 500 nucleotides, between 500 and 1000 nucleotides, between 1000 and 1500 nucleotides and greater than 1500 nucleotides. It is recognized that a reverse transcriptase may preferentially increase the possibility of generating more UMI reads from genes of one length range. It is recognized that an engineered reverse transcriptase may perform similarly, differently or comparably in a 3′-reverse transcription assay or a 5′-reverse transcription assay. It is similarly recognized that an engineered reverse transcriptase may preferentially increase the possibility of generating more UMI reads from a length of genes in a 3′-reverse transcription assay than in a 5′-reverse transcription assay.

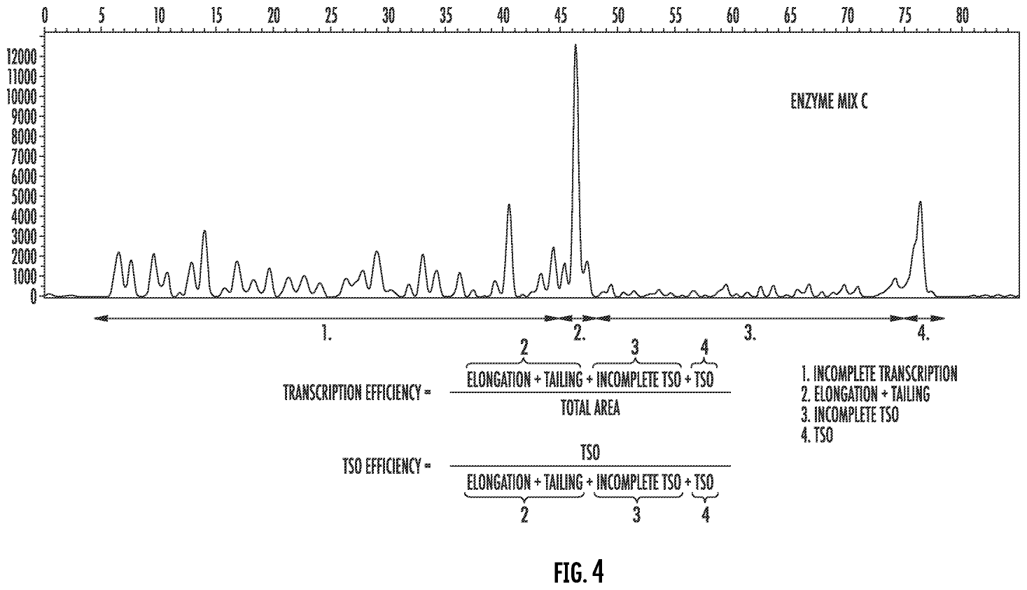

Transcription efficiency may be calculated as the sum of the area under the curve for the elongation, elongation plus tail, incomplete template switching (TSO) and complete template switching (TSO) regions over the total area under the curve for all products (see FIG. 4). Transcription efficiency reflects all those products for which transcription was successfully completed. Template switching oligonucleotide efficiency may be calculated as the area under the curve for the complete template switching region over the total area under the curve for all full-length products (see FIG. 4). An engineered reverse transcriptase may have an increased transcription efficiency, an increased TSO efficiency or both an increased transcription efficiency and an increased TSO efficiency.

The term “sequencing,” as used herein, generally refers to methods and technologies for determining the sequence of nucleotide bases in one or more polynucleotides. Any method of sequencing known in the art may be used to evaluate the products of a reaction performed by an engineered reverse transcriptase of the current application. Sequencing can be performed by various systems currently available, such as, without limitation, a sequencing system by Illumina®, Pacific Biosciences (PacBio®), Oxford Nanopore®, or Life Technologies (ion Torrent®). Alternatively, or in addition, sequencing may be performed using nucleic acid amplification, polymerase chain reaction (PCR) (e.g., digital PCR, quantitative PCR, or real time PCR), or isothermal amplification. In some examples, such systems provide sequencing reads (also “reads” herein). A read may include a string of nucleic acid bases corresponding to a sequence of a nucleic acid molecule that has been sequenced.

In one aspect, the present invention provides methods that utilize the engineered fusion reverse transcriptases described herein for nucleic acid sample processing. In one embodiment, the method comprises contacting a template ribonucleic acid (RNA) molecule with an engineered fusion reverse transcriptase to reverse transcribe the RNA molecule to a complementary DNA (cDNA) molecule. The contacting step may be in the presence of a plurality of nucleic acid barcode molecules, wherein each nucleic acid barcode molecule comprises a barcode sequence. The nucleic acid barcode molecule may further comprise a sequence configured to couple to a template RNA molecule. Suitable sequences include, without limitation, an oligo(dT) sequence, a random N-mer primer, or a target-specific primer. The nucleic acid barcode molecule may further comprise a template switching sequence. In other embodiments, the RNA molecule is a messenger RNA (mRNA) molecule. In one embodiment, a contacting step provides conditions suitable to allow the engineered reverse transcriptase to (i) transcribe the mRNA molecule into the cDNA molecule with the oligo(dT) sequence and/or (ii) perform a template switching reaction, thereby generating the cDNA molecule which comprises a barcode sequence, or a complement thereof. In another embodiment, the contacting step may occur in (i) a partition having a reaction volume (as further described herein and see e.g., U.S. Pat. Nos. 10,400,280 and 10,323,278, each of which is incorporated herein by reference in its entirety), (ii) in a bulk reaction where the reaction components (e.g., template RNA and engineered reverse transcriptase) are in solution, or (iii) on a nucleic acid array (see e.g., U.S. Pat. Nos. 10,480,022 and 10,030,261 as well as WO/2020/047005 and WO/2020/047010, each of which is incorporated herein by reference in its entirety).

In another embodiment, a method comprises providing a reaction volume which comprises an engineered fusion reverse transcriptase and a template ribonucleic acid (RNA) molecule and is considered a “low volume reaction”. The reaction volume may further comprise a plurality of nucleic acid barcode molecules, wherein each nucleic acid barcode molecule comprises a barcode sequence. In an embodiment, the contacting occurs in a reaction volume, a low volume reaction, which may be less than 1 nanoliter, less than 750 picoliters, or less than 500 picoliters. In other embodiments, the reaction volume is present in a partition, such as a droplet or well (including a microwell or a nanowell).

All publications, patents, and patent applications mentioned in this specification are herein incorporated by reference in their entireties to the same extent as if each individual publication, patent, or patent application was specifically and individually indicated to be incorporated by reference. It will be understood that the reference to the below examples is for illustration purposes only and do not limit the scope of the claims.

### EXAMPLES

### Example 1. Capillary Electrophoresis Assay Validation

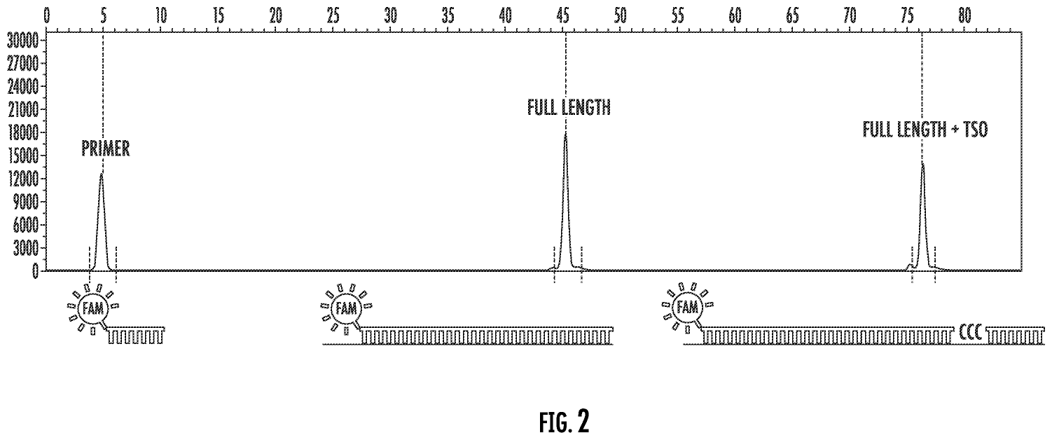

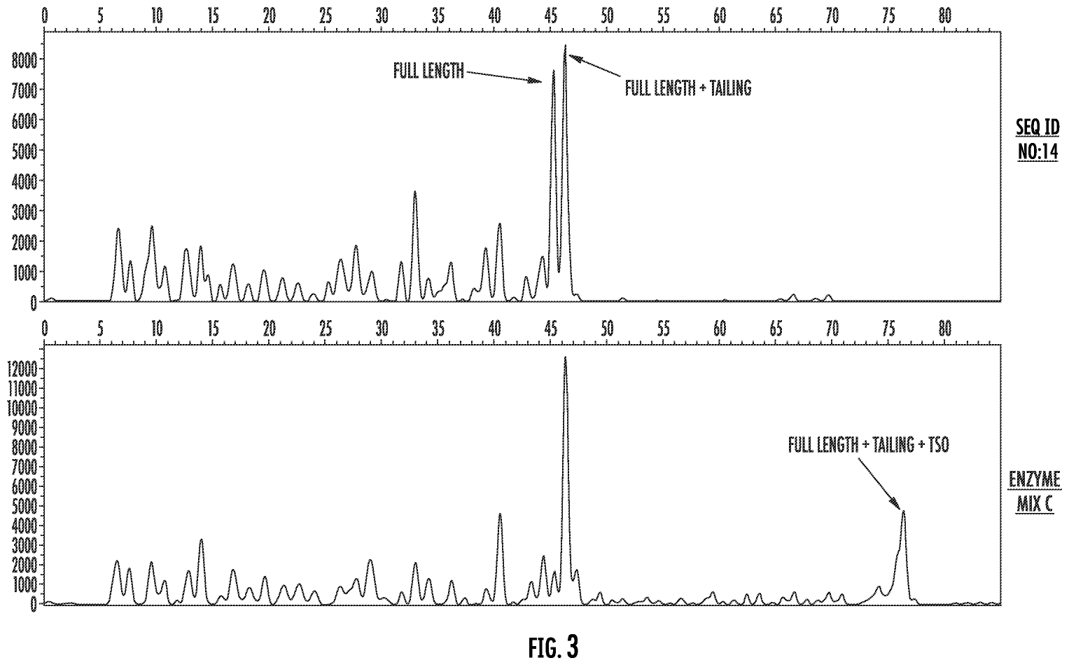

Reverse transcription and sequencing reactions were prepared. The reaction volume was 50 μl and reactions contained 5′-end labeled FAM Reverse Transcriptase primer 2, RT Reagent B (Chromium Next GEM Single Cell Reagent, 10× Genomics), RNA template (RNA Temp 2), template switching oligo 1 (TSO1), and the indicated engineered reverse transcriptase. Experimental workflow replicated that of the Chromium Single Cell Gene Expression 5′ kit (10× Genomics, Inc), except the reverse transcriptase was altered for a particular reaction. Stock concentrations and final concentrations in the reactions are shown in Table 1. Variations of the assay stock concentrations and final concentrations in the reactions shown in Table 2 were used. The reactions included stoichiometrically equal amounts of enzyme and template for single turnover conditions. Reactants were incubated at 53° C. for one hour, then diluted 1:40 in water and then 1:20 in HiDi formamide. The formamide mixture was heated to 95° C. for 5 mins, then chilled on ice for 2 mins. Samples were loaded on a Seqstudio (Thermofisher) and fragment analysis by capillary electrophoresis was carried out with the appropriate dye channels and size standards. The assay was validated with synthetically sized oligonucleotides (FIG. 2) and with a transcription positive, template switching null engineered reverse transcriptase (SEQ ID NO:14) and a transcription positive, template switching positive reverse transcriptase (Enzyme Mix C, FIG. 3). The GEM-U reagent approximates the formulation of the actual reagent mixture in a GEM assay when the contents of the Z₁ and Z₂ channels are mixed.

TABLE 1
Capillary Electrophoresis Assay Reactants and
Template, Primer and TSO sequences (SEQ ID NOS:
9-11, respectively in order of appearance.)

| | | |
|---|---|---|
| Reagent | Stock | Final |

| | | |
|---|---|---|
| RT Reagent B | 2.66x | 1.00X |

| | | | |
|---|---|---|---|
| FAM.RT.Primer2 | 100.00 | uM | 0.50 uM |
| RNA.Temp2.CE | 84.4 | uM | 0.50 uM |
| TSO1.Oligo | 91.20 | uM | 5.00 uM |
| Enzyme | 15.40 | uM | 0.50 uM |

| | | |
|---|---|---|
| Water | — | — |

TABLE 2
Capillary Electrophoresis Assay Reactants and Template, Primer and TSO sequences

| RT Reagent B (2000165) | 4.00 | x | 1.00 | x | 9.54 | uL | 76.34 | uL |
|---|---|---|---|---|---|---|---|---|
| FAM.RT.Primer2 (Variable) | 100.00 | uM | 0.5000 | uM | 0.250 | uL | 2.000 | uL |
| RNA.Temp2.CE (Variable) | 84.40 | uM | 1.00 | uM | 0.59 | uL | 4.74 | uL |
| TSO1.Oligo (Variable) | 1000.00 | uM | 64.00 | uM | 3.20 | uL | 25.60 | uL |
| DTT | 1000.00 | mM | 20.00 | mM | 1.00 | uL | 8.00 | uL |
| Gel Bead Buffer (2000018) | 1.00 | x | 0.24 | x | 11.83 | uL | 94.66 | uL |
| Polyacylamide Solution (2000052) | 10% | | 0.50% | | 2.50 | uL | 20.00 | uL |
| Enzyme | 15.40 | uM | 0.50 | uM | 1.62 | uL | 12.99 | uL |
| Water | — | | — | | 19.46 | uL | 155.68 | uL |
| Total | | | | | 50.00 | ul | 400.00 | ul |

### Example 2. Construction, Cloning and Expression of Engineered Reverse Transcriptases

Some mutants were constructed using a Q5 mutagenesis kit (NEB) with mutagenic primers per manufacturing instructions. Linearized products were circularized by KLD treatment (kinase, ligase, DpN1) and cloned. Some mutants were synthesized as whole plasmids and furnished by Twist Biosciences, South San Francisco CA.

Briefly, a vector comprising the Ss07d sequence was obtained from Integrated DNA Technologies (IDT, Coralville, IA). Cloning was performed using a Gibson Assembly kit from New England Biolabs (NEB, Ipswitch, ME). Q5 polymerase was used to generate Gibson vectors. Amplification conditions were an initial denaturation at 95° C. for 2.5 minutes, 30 cycles of denature (95° C., 30 sec), a 45 sec gradient annealing and extension at 72° C. for 6 minutes, 35 sec, followed by a final extension at 72° C. for 2 minutes. Amplification reactions with multiple annealing gradient temperatures (65.2° C., 67° C., 68.5° C. and 69.6° C.) were performed. Amplification products were evaluated on a 1.2% agarose E-Gel using SYBR-Safe. Products were pooled prior to clean-up. Cloning and expression were performed in the Acella cell line from EdgeBio (San Jose, CA). Cells were selected on LB-Kanamycin plates. Ss07d N-terminal and C-terminal fusions to an engineered reverse transcriptase having the amino acid sequence set forth in SEQ ID NO:1 were obtained by screening of bacterial colonies. The sequences of the fusion proteins were confirmed. An Ss07d N-terminal fusion protein of the amino acid sequence set forth in SEQ ID NO:8 was generated; an Ss07d C-terminal fusion protein of the amino acid sequence set forth in SEQ ID NO:6 was generated. In some aspects the Sso7d fusion proteins are produced with an N-terminal 6×HisTag and thrombin cleavage site. The 6×HisTag (SEQ ID NO: 15) is used for purification purposes and removed by thrombin cleavage.

### Example 4. 5′ GEM-X Analysis of N-Terminal and C-Terminal Fusion Protein

Experiments were carried out as found in the manufacturer's instructions for the Chromium Single Cell 5′ Gene Expression Assay kit (10× Genomics).

Table 3 details the reverse transcriptases and the fusion variants that were generated in Example 3.

TABLE 3
SEQ ID NOs of MMLV enzymes used
and fusion variants generated

| SEQ | | | DNA BINDING | DBD NAME/ |
|---|---|---|---|---|
| ID NO | MMLV | 6HIS | DOMAIN (DBD) | LOCATION |
| 1 | Variant | No | No | NA |
| 2 | NA | NA | Yes | Archeal consensus |
| | | | | sequence |
| 3 | Fusion variant | Yes | SEQ ID NO: 12 | Sto7; C-terminus |
| 5 | Fusion variant | Yes | SEQ ID NO: 12 | Sto7; C-terminus |
| 6 | Fusion variant | No | SEQ ID NO: 13 | Sso7d; C-terminus |
| 7 | Wild type | No | No | NA |
| 8 | Fusion variant | No | SEQ ID NO: 13 | Sso7d; N-terminus |

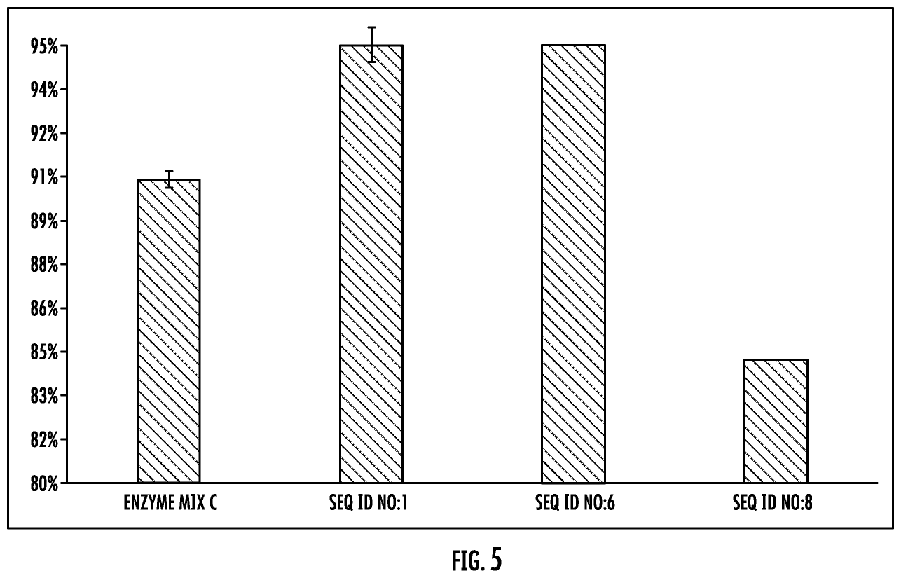

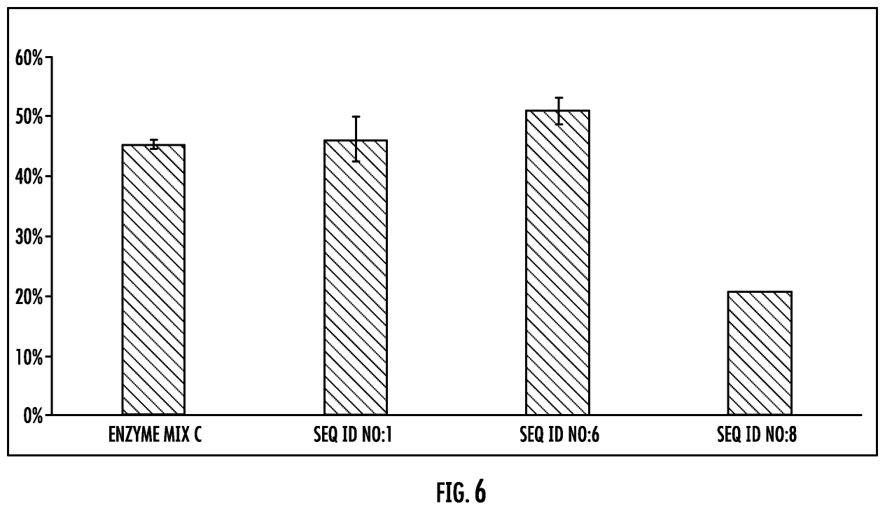

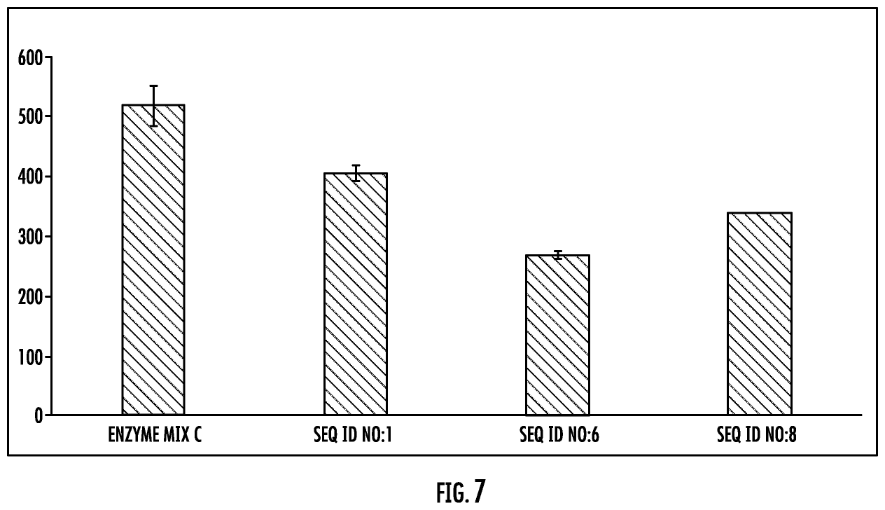

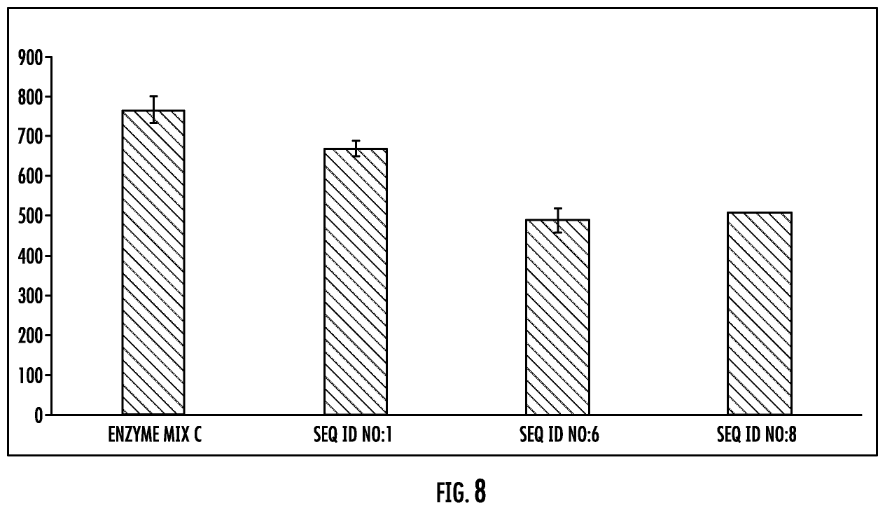

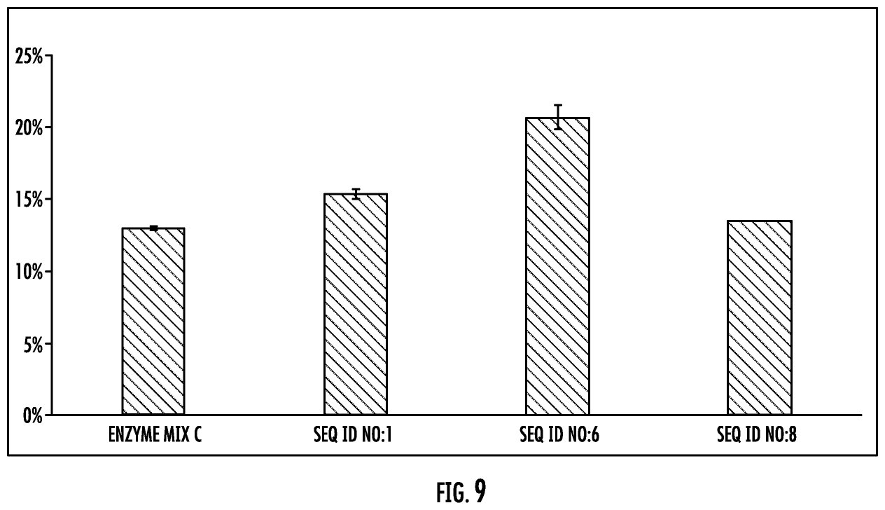

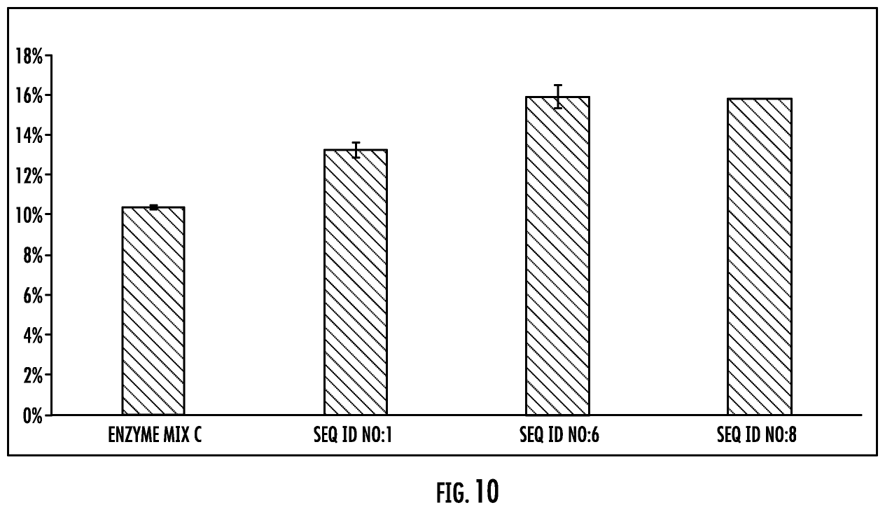

Exemplary results can be seen in FIGS. 5-10. FIG. 5 data demonstrates the increased percentage of valid barcodes read upon sequencing of products generated using one of four different RT enzyme configurations. Both SEQ ID NO: 1 and 6 demonstrated enhanced ability to incorporate barcodes into a nucleic acid product upon reverse transcription compared to the control Enzyme mix C. Conversely, SEQ ID NO: 8 was less efficient than the control enzyme mix. The same type of pattern is seen in FIG. 6 (mapped reads to transcriptome) and FIG. 9 (fraction of ribosomal protein UMI counts). FIGS. 7 and 8 reveal a different pattern of efficiency, where, while transcription products were produced by all enzymes tested, the control yielded more transcription products that results in more genes per cell and higher median UMI counts per cell, respectively, compared to SEQ ID NOS: 1, 6 or 8. FIG. 10 shows that the three variant and fusion MMLV enzymes provided products that yielded higher fraction mitochondrial UMI counts compared to the Enzyme mix C control.

As such, the data demonstrate that the SEQ ID NOs: 1, 6 and 8 were comparable to the control reverse transcriptase in a variety of experiments, and in many cases were equivalent or exceeded the activity of the control reverse transcriptase.

### Example 5. Transcription Efficiency and Template Switching Efficiency Analysis

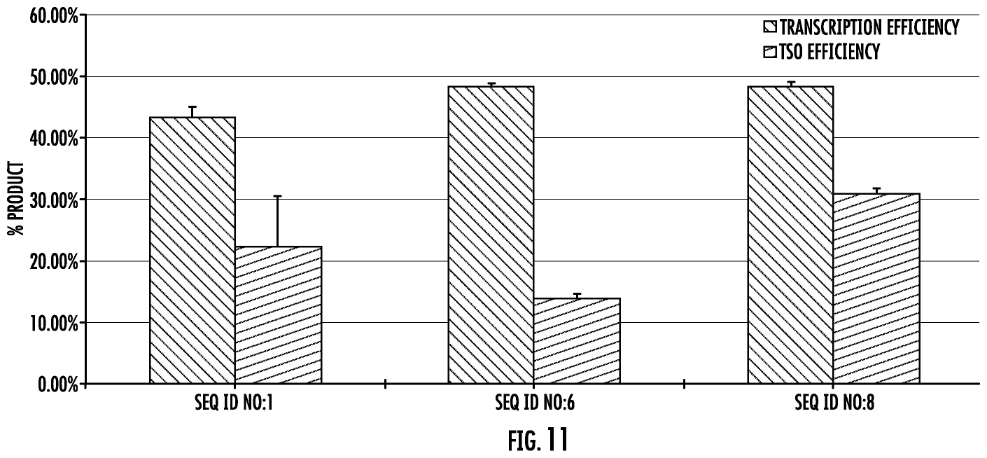

Capillary electrophoretic reactions were performed generally as described above in the previous examples, using a variety of reverse transcriptases and engineered reverse transcriptases as found in Table 3. The transcription efficiency and template switching efficiency as a percent product were determined via calculations as found on FIG. 4. FIG. 11 shows the results of an exemplary set of experiments for determining transcription efficiency and template switching efficiency of three reverse transcriptase enzymes; SEQ ID NO: 1, SEQ ID NO: 6 and SEQ ID NO: 8. As shown, the transcription efficiencies of the clones is comparable, whereas the TSO efficiency is variable from one clone to the next.

### Example 6. Template Switching Efficiency Analysis

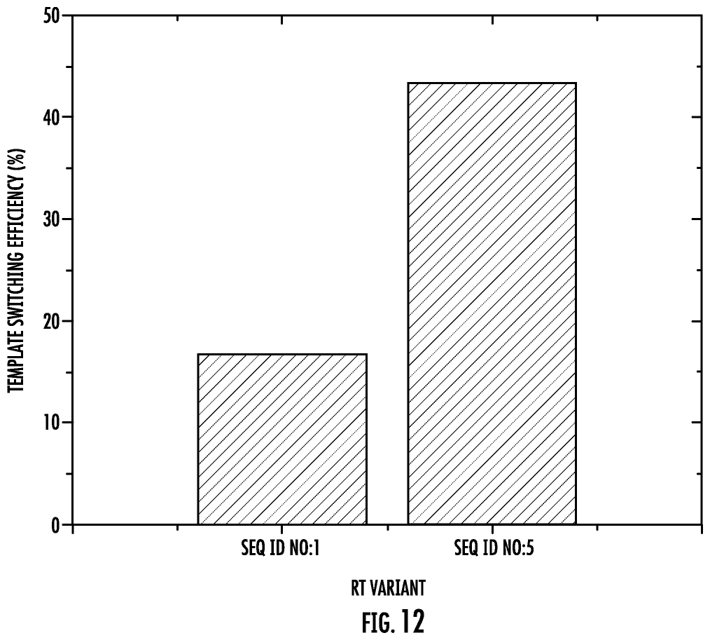

A reverse transcriptase having the amino acid sequence set forth in SEQ ID NO:1 and an engineered reverse transcriptase comprising an amino acid sequence set forth in SEQ ID NO:5 were evaluated for template switching efficiency. Results from one such series of experiments are shown in FIG. 12, where the RT from SEQ ID NO: 5 showed enhanced TSO comparative to the MMLV variant of SEQ ID NO: 1.

TABLE 3
Sequence Information

| | |
|---|---|
| SEQ ID NO: 1 | MTWLSDFPQAWAETGGMGLAVRQAPLIIPLKATSTPVSIKQYPMSQKARLGIKPHIQRL |
| | LDQGILVPCQSPWNTPLLPVKKPGTNDYRPVQDLREVNKRVEDIHPTVPNPYNLLSGPP |
| | PSHQWYTVLDLKDAFFCLRLHPTSQPLFAFEWRDPEMGISGQLTWTRLPQGFKNSPTLF |
| | NEALHRDLADFRIQHPDLILLQYVDDLLLAATSELDCQQGTRALLQTLGNLGYRASAKKA |
| | QICQKQVKYLGYLLKEGQRWLTEARKETVMGQPTPKTPRQLRRFLGKAGFCRLFIPGFAE |
| | MAAPLYPLTKPGTLFNWGPDQQKAYQEIKQALLTAPALGLPDLTKPFELFVDEKQGYAK |
| | GVLTQKLGPWRRPVAYLSKKLDPVAAGWPPCLRMVAAIAVLTKDAGKLTMGQPLVIGA |
| | PHAVEALVKQPAGRWLSKARMTHYQALLLDTDRVQFGPVVALNPATLLPLPEEGLQHN |
| | CLDILAEAHGTRPDLTDQPLPDADHTWYTNGSSLLQEGQRKAGAAVTTETEVIWAKALP |
| | AGTSAQRAELIALTQALKMAEGKKLNVYTDSRYAFATAHIHGEIYRRRGWLTSKGKEIKN |
| | KDEILALLKALFLPKRLSIIHCPGHQKGHSAEARGNRMADQAARKAAITETPDTSTLLIENS |
| | SPNSRLIN |
| SEQ ID NO: 2 | MXXXXXFKYKGXXXXVDXSKXKKVWXVGKMXSFTXDXXXGKTGRGAVSEKDAPKELXX |
| | XXXXXXXXXK |
| SEQ ID NO: 3 | MGSSHHHHHHSSGLVPRGSTWLSDFPQAWAETGGMGLAVRQAPLIIPLKATSTPVSIK |
| | QYPMSQKARLGIKPHIQRLLDQGILVPCQSPWNTPLLPVKKPGTNDYRPVQDLREVNKR |
| | VEDIHPTVPNPYNLLSGPPPSHQWYTVLDLKDAFFCLRLHPTSQPLFAFEWRDPEMGIS |
| | GQLTWTRLPQGFKNSPTLFNEALHRDLADFRIQHPDLILLQYVDDLLLAATSELDCQQGT |
| | RALLQTLGNLGYRASAKKAQICQKQVKYLGYLLKEGQRWLTEARKETVMGQPTPKTPR |
| | QLRRFLGKAGFCRLFIPGFAEMAAPLYPLTKPGTLFNWGPDQQKAYQEIKQALLTAPALG |
| | LPDLTKPFELFVDEKQGYAKGVLTQKLGPWRRPVAYLSKKLDPVAAGWPPCLRMVAAIA |
| | VLTKDAGKLTMGQPLVIGAPHAVEALVKQPAGRWLSKARMTHYQALLLDTDRVQFGP |
| | VVALNPATLLPLPEEGLQHNCLDILAEAHGTRPDLTDQPLPDADHTWYTNGSSLLQEGQ |
| | RKAGAAVTTETEVIWAKALPAGTSAQRAELIALTQALKMAEGKKLNVYTDSRYAFATAHI |
| | HGEIYRRRGWLTSKGKEIKNKDEILALLKALFLPKRLSIIHCPGHQKGHSAEARGNRMAD |
| | QAARKAAITETPDTSTLLIENSSPNSRLINGGGSMVTVKFKYKGEELEVDISKIKKVWRVG |
| | KMISFTYDDNGKTGRGAVSEKDAPKELLQMLEKSGKK |
| SEQ ID NO: 4 | TTTTTTTTTT |
| SEQ ID NO: 5 | MGSSHHHHHHSSGLVPRGSTWLSDFPQAWAETGGMGLAVRQAPLIPLKATSTPVSIK |
| | QYPMSQKARLGIKPHIQRLLDQGILVPCQSPWNTPLLPVKKPGTNDYRPVQDLREVNKR |
| | VEDIHPTVPNPYNLLSGPPPSHQWYTVLDLKDAFFCLRLHPTSQPLFAFEWRDPEMGIS |
| | GQLTWTRLPQGFKNSPTLFNEALHRDLADFRIQHPDLILLQYVDDLLLAATSELDCQQGT |
| | RALLQTLGNLGYRASAKKAQICQKQVKYLGYLLKEGQRWLTEARKETVMGQPTPKTPR |
| | QLRRFLGKAGFCRLFIPGFAEMAAPLYPLTKPGTLFNWGPDQQKAYQEIKQALLTAPALG |
| | LPDLTKPFELFVDEKQGYAKGVLTQKLGPWRRPVAYLSKKLDPVAAGWPPCLRMVAAIA |
| | VLTKDAGKLTMGQPLVIGAPHAVEALVKQPAGRWLSKARMTHYQALLLDTDRVQFGP |
| | VVALNPATLLPLPEEGLQHNCLDILAEAHGTRPDLTDQPLPDADHTWYTNGSSLLQEGQ |
| | RKAGAAVTTETEVIWAKALPAGTSAQRAELIALTQALKMAEGKKLNVYTDSRYAFATAHI |
| | HGEIYRRRGWLTSKGKEIKNKDEILALLKALFLPKRLSIIHCPGHQKGHSAEARGNRMAD |
| | QAARKAAITETPDTSTLLIENSSPNSRLINGGGSMVTVKFKYKGEEKEVDISKIKKVWRV |
| | GKMISFTYDDNGKTGRGAVSEKDAPKELLQMLEKSGKK |
| SEQ ID NO: 6 | MTWLSDFPQAWAETGGMGLAVRQAPLIIPLKATSTPVSIKQYPMSQKARLGIKPHIQRL |
| | LDQGILVPCQSPWNTPLLPVKKPGTNDYRPVQDLREVNKRVEDIHPTVPNPYNLLSGPP |
| | PSHQWYTVLDLKDAFFCLRLHPTSQPLFAFEWRDPEMGISGQLTWTRLPQGFKNSPTLF |
| | NEALHRDLADFRIQHPDLILLQYVDDLLLAATSELDCQQGTRALLQTLGNLGYRASAKKA |
| | QICQKQVKYLGYLLKEGQRWLTEARKETVMGQPTPKTPRQLRRFLGKAGFCRLFIPGFAE |
| | MAAPLYPLTKPGTLFNWGPDQQKAYQEIKQALLTAPALGLPDLTKPFELFVDEKQGYAK |
| | GVLTQKLGPWRRPVAYLSKKLDPVAAGWPPCLRMVAAIAVLTKDAGKLTMGQPLVIGA |
| | PHAVEALVKQPAGRWLSKARMTHYQALLLDTDRVQFGPVVALNPATLLPLPEEGLQHN |
| | CLDILAEAHGTRPDLTDQPLPDADHTWYTNGSSLLQEGQRKAGAAVTTETEVIWAKALP |
| | AGTSAQRAELIALTQALKMAEGKKLNVYTDSRYAFATAHIHGEIYRRRGWLTSKGKEIKN |
| | KDEILALLKALFLPKRLSIIHCPGHQKGHSAEARGNRMADQAARKAAITETPDTSTLLIENS |
| | SPNSRLINGMATVKFKYKGEEKEVDISKIKKVWRVGKMISFTYDEGGGKTGRGAVSEK |
| | DAPKELLQMLEKQKK |
| SEQ ID NO: 7 | TLNIEDEHRLHETSKEPDVSLGSTWLSDFPQAWAETGGMGLAVRQAPLIIPLKATSTPVSI |
| | KQYPMSQEARLGIKPHIQRLLDQGILVPCQSPWNTPLLPVKKPGTNDYRPVQDLREVNK |
| | RVEDIHPTVPNPYNLLSGLPPSHQWYTVLDLKDAFFCLRLHPTSQPLFAFEWRDPEMGIS |
| | GQLTWTRLPQGFKNSPTLFDEALHRDLADFRIQHPDLILLQYVDDLLLAATSELDCQQGT |
| | RALLQTLGNLGYRASAKKAQICQKQVKYLGYLLKEGQRWLTEARKETVMGQPTPKTPR |
| | QLREFLGTAGFCRLWIPGFAEMAAPLYPLTKTGTLFNWGPDQQKAYQEIKQALLTAPAL |
| | GLPDLTKPFELFVDEKQGYAKGVLTQKLGPWRRPVAYLSKKLDPVAAGWPPCLRMVAAI |
| | AVLTKDAGKLTMGQPLVILAPHAVEALVKQPPDRWLSNARMTHYQALLLDTDRVQFGP |
| | VVALNPATLLPLPEEGLQHNCLDILAEAHGTRPDLTDQPLPDADHTWYTDGSSLLQEGQ |
| | RKAGAAVTTETEVIWAKALPAGTSAQRAELIALTQALKMAEGKKLNVYTDSRYAFATAHI |
| | HGEIYRRRGLLTSEGKEIKNKDEILALLKALFLPKRLSIIHCPGHQKGHSAEARGNRMADQ |
| | AARKAAITETPDTSTLL |
| SEQ ID NO: 8 | MATVKFKYKGEEKEVDISKIKKVWRVGKMISFTYDEGGGKTGRGAVSEKDAPKELLQ |
| | MLEKQKKGSTWLSDFPQAWAETGGMGLAVRQAPLIIPLKATSTPVSIKQYPMSQKARL |
| | GIKPHIQRLLDQGILVPCQSPWNTPLLPVKKPGTNDYRPVQDLREVNKRVEDIHPTVPNP |
| | YNLLSGPPPSHQWYTVLDLKDAFFCLRLHPTSQPLFAFEWRDPEMGISGQLTWTRLPQG |
| | FKNSPTLFNEALHRDLADFRIQHPDLILLQYVDDLLLAATSELDCQQGTRALLQTLGNLGY |
| | RASAKKAQICQKQVKYLGYLLKEGQRWLTEARKETVMGQPTPKTPRQLRRFLGKAGFCR |
| | LFIPGFAEMAAPLYPLTKPGTLFNWGPDQQKAYQEIKQALLTAPALGLPDLTKPFELFVD |
| | EKQGYAKGVLTQKLGPWRRPVAYLSKKLDPVAAGWPPCLRMVAAIAVLTKDAGKLTM |
| | GQPLVIGAPHAVEALVKQPAGRWLSKARMTHYQALLLDTDRVQFGPVVALNPATLLPL |
| | PEEGLQHNCLDILAEAHGTRPDLTDQPLPDADHTWYTNGSSLLQEGQRKAGAAVTTET |
| | EVIWAKALPAGTSAQRAELIALTQALKMAEGKKLNVYTDSRYAFATAHIHGEIYRRRGWL |
| | TSKGKEIKNKDEILALLKALFLPKRLSIIHCPGHQKGHSAEARGNRMADQAARKAAITETP |
| | DTSTLLIENSSPNSRLIN |
| SEQ ID NO: 9 | ACGACCGUCG UCAUGUAGCG UUUGUCGGAG ACUCCUAGAU CAGAUGUCCU |
| | CCUGGCUACU GCA |
| SEQ ID | CGACTCACTG ACACTCGC |
| NO: 10 | |
| SEQ ID | AAGCAGTGGT ATCAACGCAG AGTACATrGrGrG |
| NO: 11 | |
| SEQ ID | MVTVKFKYKGEEKEVDISKIKKVWRVGKMISFTYDDNGKTGRGAVSEKDAPKELLQMLE |
| NO: 12 | KSGKK |
| SEQ ID | MATVKFKYKGEEKEVDISKIKKVWRVGKMISFTYDEGGGKTGRGAVSEKDAPKELLQML |
| NO: 13 | EKQKK |
| SEQ ID | MTWLSDFPQAWAETGGMGLAVRQAPLIIPLKATSTPVSIKQYPLSQEARLGIKPHIQRLL |
| NO: 14 | DQGILVPCQSPWNTPLLPVKKPGTNDYRPVQDLREVNKRVEDIHPTVPNPYNLLSGPPP |
| | SHQWYTVLDLKDAFFCLRLHPTSQPLFAFEWRDPEMGISGQLTWTRLPQGFKNSPTLFN |
| | EALHRDLADFRIQHPDLILLQYVDDLLLAATSELDCQQGTRALLQTLGNLGYRASAKKAQI |
| | CQKQVKYLGYLLKEGQRWLTEARKETVMGQPTPKTPRQLREFLGTAGFCRLWIPGFAE |
| | MAAPLYPLTKPGTLFNWGPDQQKAYQEIKQALLTAPALGLPDLTKPFELFVDEKQGYAK |
| | GVLTQKLGPWRRPVAYLSKKLDPVAAGWPPCLRMVAAIAVLTKDAGKLTMGQPLVIKA |
| | PHAVEALVKQPPDRWLSNARMTHYQALLLDTDRVQFGPVVALNPATLLPLPEEGLQHN |
| | CLDILAEAVGTRPDLTDQPLPDADHTWYTNGSSLLQEGQRKAGAAVTTETEVIWAKALP |
| | AGTSAQRAELIALTQALKMAEGKKLNVYTDSRYAFATAHIHGEIYRRRGWLTSKGKEIKN |
| | KDEILALLKALFLPKRLSIIYCPGHQKGHSAEARGNRMADQAARKAAITETPDTSTLLIENS |
| | SPNSRLIN |

1. An engineered fusion reverse transcriptase comprising:
(a) at least one archaeal DNA binding domain; and
(b) an engineered reverse transcriptase having an amino acid sequence that is at least 90% identical to SEQ ID NO:1, wherein said engineered reverse transcriptase comprises an L435 mutation, a D449 mutation, a D524 mutation, and an E607 mutation, as indexed to SEQ ID NO:7.

2. The engineered fusion reverse transcriptase of claim 1, wherein said engineered fusion reverse transcriptase exhibits an altered reverse transcriptase related activity as compared to a reverse transcriptase having the amino acid sequence set forth in SEQ ID NO:1, and wherein the altered reverse transcriptase related activity is selected from the group consisting of an altered processivity, an altered template switching efficiency, an altered transcription efficiency, an altered ability to yield mitochondrial UMI counts, an altered ability to yield ribosomal UMI counts, and an altered chemical tolerance.

3. The engineered fusion reverse transcriptase of claim 1, wherein the at least one archaeal DNA binding domain is located at the C-terminus or at the N-terminus of the engineered fusion reverse transcriptase.

4. The engineered fusion reverse transcriptase of claim 1, wherein the amino acid sequence of the at least one archaeal DNA binding domain:
(a) comprises a DNA binding domain consensus motif set forth in SEQ ID NO:2;
(b) has been altered to reduce RNAase activity; or
(c) comprises a DNA binding domain consensus motif set forth in SEQ ID NO:2 and has been altered to reduce RNAase activity.

5. The engineered fusion reverse transcriptase of claim 1, wherein the at least one archaeal DNA binding domain:
(a) is an archaeal DNA binding domain of a molecule selected from the group consisting of Sto7d, Sso7d, Sis7b, Sis7a, Ssh7b, Sto7, Aho7C, Aho7B, Aho7A, Mcu7, Mse7, Sac7e, and Sac7d;
(b) is a single-stranded DNA binding domain; or
(c) exhibits reduced RNAase activity.

6.-8. (canceled)

9. The engineered reverse transcriptase of claim 4, wherein the alteration to the amino acid sequence of the at least one archaeal DNA binding domain is selected from the group consisting of a K13 mutation, a K13L mutation, a D36 mutation, and a D36L mutation in SEQ ID NO: 2, SEQ ID NO: 12, or SEQ ID NO: 13.

10. The engineered fusion reverse transcriptase of claim 1, wherein:
(a) the at least one archaeal DNA binding domain is a Sto7 DNA binding domain, and
(b) the Sto7 DNA binding domain is located at the C-terminus of the engineered fusion reverse transcriptase.

11. The engineered fusion reverse transcriptase of claim 1, wherein the engineered fusion reverse transcriptase comprises:
(a) an E69 mutation, an L139 mutation, a D200 mutation, an E302 mutation, a T306 mutation, a W313 mutation, a T330 mutation, an L435 mutation, a P448 mutation, a D449 mutation, an N454 mutation, a D524 mutation, an L603 mutation, and an E607 mutation, indexed to SEQ ID NO:7;
(b) an E69K mutation, an L139P mutation, a D200N mutation, an E302R mutation, a T306K mutation, a W313F mutation, a T330P mutation, an L435G mutation, a P448A mutation, a D449G mutation, an N454K mutation, a D524N mutation, an L603W mutation, and an E607K mutation, indexed to SEQ ID NO:7;
(c) the amino acid sequence of SEQ ID NO: 1: or
(d) the amino acid sequence set forth in SEQ ID NO: 3, SEQ ID NO: 5, SEQ ID NO: 6, or SEQ ID NO: 8.

12. The engineered fusion reverse transcriptase of claim 1, wherein the engineered reverse transcriptase further comprises at least one mutation selected from the group consisting of:
(a) an M39 mutation, an M66 mutation, a D653 mutation, and an L671 mutation: or
(b) an M39V mutation and an M66L mutation,
wherein said mutation is indexed to an amino acid sequence set forth in SEQ ID NO:7.

13-14. (canceled)

15. The engineered fusion reverse transcriptase of claim 2, wherein the altered template switching efficiency is an increased template switching efficiency and wherein the increased template switching efficiency is at least 0.5× greater than the template switching efficiency exhibited by an engineered reverse transcriptase having the amino acid sequence set forth in SEQ ID NO:1.

16. The engineered fusion reverse transcriptase of claim 1, wherein the engineered fusion reverse transcriptase comprises at least two archaeal DNA binding domains.

17. The engineered fusion reverse transcriptase of claim 16, wherein:
(a) at least one of the at least two archaeal DNA binding domains is located at the N-terminus of the engineered fusion reverse transcriptase and at least one of the at least two archaeal DNA binding domains is located at the C-terminus of the engineered fusion reverse transcriptase; or
(b) the at least two archaeal DNA binding domains are located at the C-terminus or the N-terminus of the engineered fusion reverse transcriptase.

18. (canceled)

19. The engineered fusion reverse transcriptase of claim 17, wherein:
(a) the at least one archaeal DNA binding domain located at the N-terminus of the engineered fusion reverse transcriptase is Sso7d and the at least one archaeal DNA binding domain located at the C-terminus of the engineered fusion reverse transcriptase is Sso7d;
(b) the at least one archaeal DNA binding domain located at the N-terminus of the engineered fusion reverse transcriptase is Sto7 and the at least one archaeal DNA binding domain located at the C-terminus of the engineered fusion reverse transcriptase is Sto7;
(c) the at least one archaeal DNA binding domain located at the N-terminus of the engineered fusion reverse transcriptase is Ss07d and the at least one archaeal DNA binding domain located at the C-terminus of the engineered fusion reverse transcriptase is Sto7; or
(d) the at least one archaeal DNA binding domain located at the N-terminus of the engineered fusion reverse transcriptase is Sto7 and the at least one archaeal DNA binding domain located at the C-terminus of the engineered fusion reverse transcriptase is Ss07d.

20. (canceled)

21. The engineered fusion reverse transcriptase of claim 2, wherein: the altered reverse transcriptase related activity is:
(a) an increased transcription efficiency; or
(b) an increased transcription efficiency and an increased template switching efficiency.

22.-26. (canceled)

27. The engineered fusion reverse transcriptase of claim 1, wherein said engineered reverse transcriptase comprises the amino acid sequence of SEQ ID NO: 1 or an amino acid sequence that is at least 95% identical to SEQ ID NO:1, and wherein the engineered reverse transcriptase further comprises a combination of mutations indexed to SEQ ID NO:7, and wherein the combination of mutations comprises at least one mutation selected from the group consisting of:
(a) an A32 mutation, an M39 mutation, an M66 mutation, an L72 mutation, an F155 mutation, an E201 mutation, a G248 mutation, an E286 mutation, a T287 mutation, a Y344 mutation, a 1347 mutation, a W388 mutation, an R411 mutation, an H503 mutation, an H594 mutation, an H634 mutation, a G637 mutation, an H638 mutation, a D653 mutation, and an L671 mutation;
(b) an M39V mutation, an M66L mutation, an F155Y mutation, an E201Q mutation, a T287A mutation, an R411F mutation, an H503V mutation, an H594K mutation, an H634Y mutation, a G637R mutation and an H638G mutation;
(c) an A32V mutation, an L72R mutation, a D200C mutation, a G248C mutation, an E286R mutation, and a W388R mutation; and
(d) a Y344L mutation and an I347L mutation.

28. A method for performing a reverse transcription reaction for generating a nucleic acid product from an RNA template using the engineered fusion reverse transcriptase from claim 1.

29. The method of claim 28, wherein the method comprises using the engineered fusion reverse transcriptase of claim 11.

30. The method of claim 28, wherein the engineered fusion reverse transcriptase comprises a DNA binding domain comprising a mutation selected from the group consisting of a K13 mutation, a K13L mutation, a D36 mutation, and a D36L in SEQ ID NO: 2, SEQ ID NO: 12, or SEQ ID NO: 13.

31. The method of claim 28, wherein the engineered fusion reverse transcriptase comprises the amino acid sequence of SEQ ID NO: 3, SEQ ID NO: 5, SEQ ID NO: 6, or SEQ ID NO: 8.

32. A method for performing a reverse transcription reaction for generating a nucleic acid product from an RNA template using an engineered fusion reverse transcriptase, wherein the engineered fusion reverse transcriptase comprises:
(a) at least one archaeal DNA binding domain; and
(b) an engineered reverse transcriptase,
wherein the amino acid sequence of the engineered reverse transcriptase comprises an M39 mutation, an L435 mutation, a D449 mutation, a D524 mutation, an E607 mutation, a D653 mutation and an L671 mutation as indexed to SEQ ID NO:7, and further comprises a second combination of mutations indexed to SEQ ID NO:7 selected from the group consisting of:
(i) an E69K mutation, an E302R mutation, a T306K mutation, a W313F mutation, an L435G mutation, and an N454K mutation, and further comprising at least one mutation selected from the group consisting of an M39V mutation, an M66L mutation, an L139P mutation, an F155Y mutation, a D200N mutation, an E201Q mutation, a T287A mutation, a T330P mutation, an R411F mutation, a P448A mutation, a D449G mutation, an H503V mutation, an H594K mutation, L603W mutation, an E607K mutation, an H634Y mutation, a G637R mutation and an H638G mutation;
(ii) an L139P mutation, a D200N mutation, a T330P mutation, an L603W mutation, and an E607K mutation, and further comprising at least one mutation selected from the group consisting of: an M39V mutation, an M66L mutation, an E69K mutation, an F155Y mutation, an E2010 mutation, a T287A mutation, an E302R mutation, a T306K mutation, a W313F mutation, an R411F mutation, an L435G mutation, a P448A mutation, a D449G mutation, an N454K mutation, an H503V mutation, an H594K mutation, an H634Y mutation, a G637R mutation and an H638G mutation;
(iii) an A32V mutation, an L72R mutation, a D200C mutation, a G248C mutation, an E286R mutation, an E302R mutation, a W388R mutation, and an L435G mutation; and
(iv) a Y344L mutation and an I347L mutation.

33.-35. (canceled)


SEQUENCE LISTING (15 sequences)

SEQ ID NO: 1 - protein, 661 aa, synthetic construct
  REGION 1..661: note = Description of Artificial Sequence: Synthetic polypeptide
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 2 - protein, 68 aa, synthetic construct
  REGION 1..68: note = Description of Artificial Sequence: Synthetic polypeptide
  VARIANT 2..6: note = Any amino acid
  VARIANT 12..15: note = Any amino acid
  VARIANT 18: note = Any amino acid
  VARIANT 21: note = Any amino acid
  VARIANT 26: note = Any amino acid
  VARIANT 31: note = Any amino acid
  VARIANT 35: note = Any amino acid
  VARIANT 37..39: note = Any amino acid
  VARIANT 57..67: note = Any amino acid
MXXXXXFKYKGXXXXVDXSKXKKVWXVGKMXSFTXDXXXGKTGRGAVSEKDAPKELXXXXXXXXXXXK

SEQ ID NO: 3 - protein, 747 aa, synthetic construct
  REGION 1..747: note = Description of Artificial Sequence: Synthetic polypeptide
MGSSHHHHHHSSGLVPRGSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGGGSMVTVKFKYKGEELEVDISKIKKVWRVGKMISFTYDDNGKTGRGAVSEKDAPKELLQMLEKSGKK

SEQ ID NO: 4 - DNA, 10 bp, synthetic construct
  misc_feature 1..10: note = Description of Artificial Sequence: Synthetic oligonucleotide
  source 1..10: mol_type = other DNA
tttttttttt

SEQ ID NO: 5 - protein, 747 aa, synthetic construct
  REGION 1..747: note = Description of Artificial Sequence: Synthetic polypeptide
MGSSHHHHHHSSGLVPRGSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGGGSMVTVKFKYKGEEKEVDISKIKKVWRVGKMISFTYDDNGKTGRGAVSEKDAPKELLQMLEKSGKK

SEQ ID NO: 6 - protein, 726 aa, synthetic construct
  REGION 1..726: note = Description of Artificial Sequence: Synthetic polypeptide
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGMATVKFKYKGEEKEVDISKIKKVWRVGKMISFTYDEGGGKTGRGAVSEKDAPKELLQMLEKQKK

SEQ ID NO: 7 - protein, 671 aa, unidentified
  REGION 1..671: note = Description of Unknown: Moloney murine leukemia virus sequence
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLL

SEQ ID NO: 8 - protein, 726 aa, synthetic construct
  REGION 1..726: note = Description of Artificial Sequence: Synthetic polypeptide
MATVKFKYKGEEKEVDISKIKKVWRVGKMISFTYDEGGGKTGRGAVSEKDAPKELLQMLEKQKKGSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 9 - RNA, 63 nt, synthetic construct
  misc_feature 1..63: note = Description of Artificial Sequence: Synthetic oligonucleotide
  source 1..63: mol_type = other RNA
acgaccgtcgtcatgtagcgtttgtcggagactcctagatcagatgtcctcctggctactgca

SEQ ID NO: 10 - DNA, 18 bp, synthetic construct
  misc_feature 1..18: note = Description of Artificial Sequence: Synthetic primer
  source 1..18: mol_type = other DNA
cgactcactgacactcgc

SEQ ID NO: 11 - DNA, 30 bp, synthetic construct
  misc_feature 1..30: note = Description of Artificial Sequence: Synthetic oligonucleotide
  source 1..30: mol_type = other DNA
aagcagtggtatcaacgcagagtacatggg

SEQ ID NO: 12 - protein, 64 aa, synthetic construct
MVTVKFKYKGEEKEVDISKIKKVWRVGKMISFTYDDNGKTGRGAVSEKDAPKELLQMLEKSGKK

SEQ ID NO: 13 - protein, 64 aa, Sulfolobus solfataricus
MATVKFKYKGEEKEVDISKIKKVWRVGKMISFTYDEGGGKTGRGAVSEKDAPKELLQMLEKQKK

SEQ ID NO: 14 - protein, 661 aa, synthetic construct
  REGION 1..661: note = Description of Artificial Sequence: Synthetic polypeptide
MTWLSDFPQAWAETGGMGLAVRQAPLIIPLKATSTPVSIKQYPLSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSNARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 15 - protein, 6 aa, synthetic construct
  REGION 1..6: note = Description of Artificial Sequence: Synthetic 6xHis tag
HHHHHH